SEQUENCE LISTING (42 sequences)

SEQ ID NO: 1 - protein, 10 aa, Artificial Sequence
  Lysine tail (short), synthetic
ASSAGSKGSK

SEQ ID NO: 2 - protein, 12 aa, Artificial Sequence
  Lysine tail (medium), synthetic
ASSAFGSKGKSK

SEQ ID NO: 3 - protein, 14 aa, Artificial Sequence
  Lysine tail (long), synthetic
ASSAGSKGKSKGSK

SEQ ID NO: 4 - protein, 164 aa, Artificial Sequence
  iMab100+VSV+HIS sequence, synthetic
MNVKLVEKGGNFVENDDDLKLTCRAEGYTIGPYCMGWFRQAPNDDSTNVATINMGGGITYYGDSVKERFDIRRDNASNTVTLSMDDLQPEDSAEYNCAGDSTIYASYYECGHGLSTGGYGYDSHYRGQGTDVTVSSASSAGGGGSYTDIEMNRLGKSHHHHHHG

SEQ ID NO: 5 - protein, 162 aa, Artificial Sequence
  iMab100 135-02-0001, synthetic
MNVQLVESGGNFVENDQDLSLTCRASGYTIGPYCMGWFRQAPNQDSTGVATINMGGGITYYGDSVKERFRIRRDNASNTVTLSMQNLQPQDSANYNCAADSTIYASYYECGHGLSTGGYGYDSRGQGTSVTVSSASSAGGGGSYTDIEMNRLGKSHHHHHHG

SEQ ID NO: 6 - protein, 162 aa, Artificial Sequence
  iMab 136-02-0001, synthetic
MNVKLVEKGGNFVENDDDLRLTCRAEGYTIGPYCMGWFRQAPNRDSTNVATINMGGGITYYGDSVKERFDIRRDNASNTVTLSMTNLQPSDSASYNCAADSTIYASYYECGHGLSTGGYGYDSRGQGTRVTVSSASSAGGGGSYTDIEMNRLGKSHHHHHHG

SEQ ID NO: 7 - protein, 162 aa, Artificial Sequence
  iMab 137-02-0001 sequence, synthetic
MNVQLVESGGNFVENDQSLSLTCRASGYTIGPYCMGWFRQAPNSRSTGVATINMGGGITYYGDSVKGRFTIRRDNASNTVTLSMNDLQPRDSAQYNCAADSTIYASYYECGHGLSTGGYGYDSRGQGTDVTVSSASSAGGGGSYTDIEMNRLGKSHHHHHHG

SEQ ID NO: 8 - protein, 162 aa, Artificial Sequence
  consensus sequence, synthetic
MNVXLVEXGGNFVENDXXLXLTCRAXGYTIGPYCMGWFRQAPNXDSTXVATINMGGGITYYGDSVKERFXIRRDNASNTVTLSMXXLQPXDSAXYNCAXDSTIYASYYECGHGLSTGGYGYDSRGQGTXVTVSSASSAGGGGSYTDIEMNRLGKSHHHHHHG

SEQ ID NO: 9 - DNA, 22 bp, Artificial Sequence
  Primer Pr8, synthetic
cctgaaacctgaggacacggcc

SEQ ID NO: 10 - DNA, 21 bp, Artificial Sequence
  Primer Pr9, synthetic
cagggtccccttgtgccccag

SEQ ID NO: 11 - DNA, 27 bp, Artificial Sequence
  Primer Pr16, synthetic
ccacrtccaccaccacrcaygtgacct

SEQ ID NO: 12 - DNA, 27 bp, Artificial Sequence
  Primer Pr49, synthetic
ggtgacctgggtacccttgtgccccgg

SEQ ID NO: 13 - DNA, 22 bp, Artificial Sequence
  Primer Pr56, synthetic
ggagcgctgagggggtctcatg

SEQ ID NO: 14 - DNA, 24 bp, Artificial Sequence
  Primer Pr73, synthetic
gaggacactgccgtatattacttg

SEQ ID NO: 15 - DNA, 24 bp, Artificial Sequence
  Primer Pr75, synthetic
gaggacactgcagaatataacttg

SEQ ID NO: 16 - DNA, 22 bp, Artificial Sequence
  Primer Pr76, synthetic
ccagggaaggcagcgctgagtt

SEQ ID NO: 17 - DNA, 22 bp, Artificial Sequence
  Primer Pr811, synthetic
gacctgggtcccaggtttccca

SEQ ID NO: 18 - DNA, 25 bp, Artificial Sequence
  Primer Pr813, synthetic
gaggacacggcaggtctataacttg

SEQ ID NO: 19 - DNA, 24 bp, Artificial Sequence
  Primer Pr814, synthetic
gaggacacggaaagctttaccttg

SEQ ID NO: 20 - DNA, 28 bp, Artificial Sequence
  Primer Pr815, synthetic
cggtgacctgggtcccctggtgtcccag

SEQ ID NO: 21 - DNA, 28 bp, Artificial Sequence
  Primer Pr816, synthetic
cggtgacctgggtcccctggtatccccg

SEQ ID NO: 22 - DNA, 26 bp, Artificial Sequence
  Primer Pr817, synthetic
cggtgacctgggtcccctgattcccg

SEQ ID NO: 23 - DNA, 28 bp, Artificial Sequence
  Primer Pr822, synthetic
cctgaggacgcggccattctattacttg

SEQ ID NO: 24 - DNA, 28 bp, Artificial Sequence
  Primer Pr823, synthetic
cctgaggccgcaggcattctattacttg

SEQ ID NO: 25 - DNA, 28 bp, Artificial Sequence
  Primer Pr824, synthetic
cctgaggctgcaggcattctataacttg

SEQ ID NO: 26 - DNA, 26 bp, Artificial Sequence
  Primer Pr829, synthetic
cggtgacctgggtcccctgttcccca

SEQ ID NO: 27 - DNA, 25 bp, Artificial Sequence
  Primer Pr830, synthetic
cggtgacctgggtccaagcttccga

SEQ ID NO: 28 - DNA, 5100 bp, Artificial Sequence
  CM126 sequence, synthetic
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatatccggatatagttcctcctttcagcaaaaaacccctcaagacccgtttagaggccccaaggggttatgctagttattgctcagcggtggcagcagccaactcagcttcctttcgggctttgttagcagccggatccttagtggtgatggtgatggtggcttttgcccaggcggttcatttctatatcggtatagctgccaccgccaccggccgagctggccgacgagacggtaacgtcggtaccctgaccacggtagtggctatcgtagccgtaaccgccggtactcaggccatgaccacattcataatagctcgcgtaaatggtagaatcacctgcacaattgtattctgcagagtcttccggttgcagatcgtccatcgataaggtaacggtgttggacgcgttgtcgcgacggatatcgaagcgctctttgacggagtcaccgtagtacgtaataccgccacccatgttgatcgtggccacgttagtactgtcgtcgttcggcgcctgacggaaccaacccatgcagtacgggccaatggtgtaaccttcagcacggcacgtgagcttaagatcgtcatcgttttcgacgaaattgccacctttttcaaccagtttcacattcatatgtatatctccttcttaaagttaaacaaaattatttctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 29 - DNA, 402 bp, Artificial Sequence
  iMab100 DNA sequence, synthetic
aatgtgaaactggttgaaaaaggtggcaatttcgtcgaaaacgatgacgatcttaagctcacgtgccgtgctgaaggttacaccattggcccgtactgcatgggttggttccgtcaggcgccgaacgacgacagtactaacgtggccacgatcaacatgggtggcggtattacgtactacggtgactccgtcaaagagcgcttcgatatccgtcgcgacaacgcgtccaacaccgttaccttatcgatggacgatctgcaaccggaagactctgcagaatacaattgtgcaggtgattctaccatttacgcgagctattatgaatgtggtcatggcctgagtaccggcggttacggctacgatagccactaccgtggtcagggtaccgacgttaccgtctcg

SEQ ID NO: 30 - protein, 135 aa, Artificial Sequence
  iMab100 protein sequence, synthetic
NVKLVEKGGNFVENDDDLKLTCRAEGYTIGPYCMGWFRQAPNDDSTNVATINMGGGITYYGDSVKERFDIRRDNASNTVTLSMDDLQPEDSAEYNCAGDSTIYASYYECGHGLSTGGYGYDSHYRGQGTDVTVSS

SEQ ID NO: 31 - DNA, 6551 bp, Artificial Sequence
  CM114-iMab 113 DNA sequence, synthetic
gatcctacctgacgctttttatcgcaactctctactgtttctccatacccgttttttgggctaacaggagaagatataccatgaaaaaactgttatttgcgattccgctggtggtgccgttttatagccatagcgcgggcggccgcaatgtgaaactggttgaaaaaggtggcaatttcgtcgaaaacgatgacgatcttaagctcacgtgccgtgctgaaggttacaccattggcccgtactccatgggttggttccgtcaggcgccgaacgacgacagtactaacgtgtcctgcatcaacatgggtggcggtattacgtactacggtgactccgtcaaagagcgcttcgatatccgtcgcgacaacgcgtccaacaccgttaccttatcgatggacgatctgcaaccggaagactctgcagaatacaattgtgcaggtgattctaccatttacgcgagctattatgaatgtggtcatggcctgagtaccggcggttacggctacgatagccactaccgtggtcagggtaccgacgttaccgtctcgtcggccagctcggccggtggcggtggcagctataccgatattgaaatgaaccgcctgggcaaaaccggcagcagtggtgattcgggcagcgcgtggagtcatccgcagtttgagaaagcggcgcgcctggaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaaggcgcgcctgtaatgaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactctagaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtatgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgagcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagactgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgttctagagttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcg

SEQ ID NO: 32 - DNA, 6551 bp, Artificial Sequence
  CM114-iMab 114 DNA sequence, synthetic
cgcgcctgtaatgaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctactctagaatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtatgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgagcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagactgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgttctagagttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggagaagatataccatgaaaaaactgttatttgcgattccgctggtggtgccgttttatagccatagcgcgggcggccgcaatgtgaaactggttgaaaaaggtggcaatttcgtcgaaaacgatgacgatcttaagctcacgtgccgtgctgaaggttacaccattggcccgtactccatgggttggttccgtcaggcgccgaacgacgacagtactaacgtggccacgatcaacatgggtggcggtattacgtactacggtgactccgtcaaagagcgcttcgatatccgtcgcgacaacgcgtccaacaccgttaccttatcgatggacgatctgcaaccggaagactctgcagaatacaattgtgcaggtgattctaccatttacgcgagctattatgaatgtggtcatggcctgagtaccggcggttacggctacgatagccactaccgtggtcagggtaccgacgttaccgtctcgtcggccagctcggccggtggcggtggcagctataccgatattgaaatgaaccgcctgggcaaaaccggcagcagtggtgattcgggcagcgcgtggagtcatccgcagtttgagaaagcggcgcgcctggaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaagg

SEQ ID NO: 33 - protein, 158 aa, Artificial Sequence
  Protein sequence for VAPs with bovine LF binding characteristics, synthetic
MNVKLVEKGGNFVENDDDLKLTCRAEGYTIGPYSMGWFRQAPNDDSTNVSCINMGGGITYYGDSVKERFDIRRDNASNTVTLSMDDLQPEDSAVYSCAADWWDGFTYGSTWYNPSSYDYRGQGTDVTVSSASSAGGGGSYTDIEMNRLGKSHHHHHHG

SEQ ID NO: 34 - protein, 163 aa, Artificial Sequence
  Protein sequence for VAPs with bovine LF binding characteristics, synthetic
MNVKLVEKGGNFVENDDDLKLTCRAEGHTIGPYSMGWFRQAPNDDSTNVSCINMGGGITYYGDSVKERFDIRRDNASNTVTLSMDDLQPEDSAVYNCAAVMVRTRIPYGSSWYLHPLNTYEYDYRGQGTDVTVSSASSAGGGGSYTDIEMNRLGKSHHHHHHG

SEQ ID NO: 35 - protein, 151 aa, Artificial Sequence
  Protein sequence for VAPs with bovine LF binding characteristics, synthetic
MNVKLVEKGGNFVENDDDLKLTCRAEGYTIGPYCMGWFRQAPNDDSTNVSCINMGGGITYYGDSVKERFDIRRDNASNTVTLSMDDLQPEDSAVYNCAADPVGSCYADGFDSRGQGTDVTVSSASSAGGGGSYTDIEMNRLGKSHHHHHHG

SEQ ID NO: 36 - protein, 129 aa, Artificial Sequence
  Protein sequence for VAPs with bovine LF binding characteristics, synthetic
MIKVYTDRENYGAVGSQVTLHCSASGYTIGPISFTWRYQPEGDRDAISIFHYNMGDGSIVIHNLDYSDNGSFTCAADPVGSCYADGFDSGNSSQVTLYVFEASSAGGGGSYTDIEMNRLGKSHHHHHHG

SEQ ID NO: 37 - protein, 129 aa, Artificial Sequence
  Protein sequence for VAPs with bovine LF binding characteristics, synthetic
MIKVYTDRENYGAVGSQVTLHCSASGYTIGPISFTWRYQPEGDRDAISIFHYNMGDGSIVIHNLDYSDNGSFTCAADPRGSCWVGEYDYGNSSQVTLYVFEASSAGGGGSYTDIEMNRLGKSHHHHHHG

SEQ ID NO: 38 - protein, 129 aa, Artificial Sequence
  Protein sequence for VAPs with bovine LF binding characteristics, synthetic
MLQVDIKPSQGEISVGESKFFLCQASGYTIGPSISWFSPNGEKLNMGSSTLTIYNANIDSAGIYNCATDGPEYGSSCQRTWVDSPDEFDFSEASVNVKIFQASSAGGGGSYTDIEMNRLGKSHHHHHHG

SEQ ID NO: 39 - DNA, 302 bp, Artificial Sequence
  iMab1300 DNA sequence, synthetic
ctgcaggttgacatcaaaccgtcccagggtgaaatctccgttggtgaatccaaattcttcctgtgccaggcttccggttacaccatcggtccgtgcatctcctggttctccccgaacggtgaaaaactgaacatgggttcctccaccctgaccatctacaacgctaacatcgacgacgctggtatctacaaatgcgctgctgactccaccatctacgcttcctactacgaatgcggtcacggtatctccaccggtggttacggttaccagtccgaagctaccgttaacgttaaaatcttcca

SEQ ID NO: 40 - DNA, 329 bp, Artificial Sequence
  iMab 1500 DNA sequence, synthetic
atcaaagtttacaccgaccgtgaaaactacggtgctgttggttcccaggttaccctgcactgctccgcttccggttacaccatcggtccgatctgcttcacctggcgttaccagccggaaggtgaccgtgacgctatctccatcttccactacaacatgggtgacggttccatcgttatccacaacctggactactccgacaacggtaccttcacctgcgctgctgactccaccatctacgcttcctactacgaatgcggtcacggtatctccaccggtggttacggttacgttggtaaaacctcccaggttaccctgtacgttttcga

SEQ ID NO: 41 - DNA, 329 bp, Artificial Sequence
  DNA sequence and adapted restiction site of iMab 143-02-0003, synthetic
atcaaagtttacaccgaccgtgaaaactacggtgctgttggttcccaggttaccctgcactgctccgcttccggttacaccatcggtccgatcagcttcacctggcgttaccagccggaaggtgaccgtgacgctatctccatcttccactacaacatgggtgacggttccatcgttatccacaacctggactactccgacaacggaagcttcacctgcgccgcagactccaccatctacgcttcctactacgaatgcggtcacggtatctccaccggtggttacggttacgttgggaattcctcccaggttaccctgtacgttttcga

SEQ ID NO: 42 - DNA, 302 bp, Artificial Sequence
  DNA sequence and adapted restiction site of iMab 144-02-0003, synthetic
ctgcaagttgacatcaaaccgtcccagggtgaaatctccgttggtgaatccaaattcttcctgtgccaggcttccggttacaccatcggtccgagcatctcctggttctccccgaacggtgaaaaactgaacatgggttcctccaccctgaccatctacaacgctaacatcgactctgcaggtatctacaaatgcgccgcagactccaccatctacgcttcctactacgaatgcggtcacggtatctccaccggtggttacggttaccagtccgaagcttccgttaacgttaaaatcttcca

(19) United States
(12) Patent Application Publication
(10) Pub. No.: US 2006/0177437 A1
(43) Pub. Date: Aug. 10, 2006

(54) Binding peptides: methods for their generation and use

(75) Inventors: Erwin Houtzager, Amerongen (NL); Wietse Willebrands, Wageningen (NL); Guy de Roo, Wageningen (NL); KeesJan Francoijs, Renkum (NL); Irma Maria Caecilia Vijn, Bennekom (NL)

Correspondence Address: TRASK BRITT, P.O. BOX 2550, SALT LAKE CITY, UT, 84110, US

(73) Assignee: CatchMabs B.V., Wageningen (NL)

(21) Appl. No.: 11/299,288

(22) Filed: Dec. 9, 2005

Related U.S. Application Data
(63) Continuation of application No. PCT/NL04/00407, filed on Jun. 9, 2004.

(30) Foreign Application Priority Data
Jun. 10, 2003 (EP) 03076792.5

(51) Int. Cl. A61K 39/395; C07K 16/10; C07K 16/12; C07K 16/40

(52) U.S. Cl. 424/133.1; 530/388.1; 530/388.3; 530/388.4; 530/388.25; 530/388.26

(57) ABSTRACT

Described are means and methods for generating binding peptide associated with a suitable core region, the resulting proteinaceous molecule and uses thereof. The invention provides a solution to the problems associated with the use of binding molecules over their entire range of use. Binding molecules can be designed to accommodate extreme conditions of use, such as extreme temperatures or pH. Alternatively, binding molecules can be designed to respond to very subtle changes in the environment.

### CROSS-REFERENCE TO RELATED APPLICATIONS

This application is a continuation of PCT International Patent Application No. PCT/NL2004/000407, filed on Jun. 9, 2004, designating the United States of America, and published, in English, as PCT International Publication No. WO 2004/108749 A2 on Dec. 16, 2004, which application claims priority to European Patent Application Serial No. 03076792.5, filed Jun. 10, 2003, the contents of each of which are hereby incorporated herein by this reference.

### TECHNICAL FIELD

The present invention relates to the field of biotechnology. The invention, in particular, relates to the generation of binding peptides and their various uses.

### BACKGROUND

Binding peptides are currently used in a wide variety of applications. Their popularity is largely due to the remarkable specificity that can be obtained using these binding peptides.

Many different types of binding peptides are being developed. For many of these, the relative small production capabilities, stability, reusability and the comparatively high production costs are a problem for wide-scale use. Recent developments have allowed the generation of low-cost binding peptides with high specificity at intermediate scales. It is expected that truly large-scale uses will come within reach in the near future. High profile purification of target molecules from complex fluids is expensive and labor intensive with classical affinity chromatographical methods. The yields and purities are often low, making most affinity systems economically unattractive. In contrast, capturing chromatography, i.e., retrieval of targets via specific binding with, e.g., antibodies, gives high yields and good qualities. However, these systems are very expensive and hardly reusable. In order to introduce capture chromatography into bulky industries, economical features like reusability, stability and good yields are important.

Milk is a very complex mixture with all sorts of molecules, like sugars, nucleic acids, proteins, etc. Most of these components are very valuable in a concentrated form but hard to purify or hard to obtain in a bioactive form. Some components are very hard to purify because of their low concentration, loss in biological activity or technical difficulties that go hand in hand with the purification methods available. The development of VAPs against specific milk or milk-derived components will enable the purification of biological active components on a large-scale basis and on economically attractive terms. Some examples of valuable components that can be obtained from milk or milk-derived streams are all lactoferrin forms, lactoperoxidases, growth factors, antibodies, lysozyme, oligosaccharides, etc., not limited to these examples.

Besides milk, other industrial, product, waste and other streams can be used to remove components therefrom. These specific components can be either valuable after purification or undesired in concentrations present in the process streams.

### DISCLOSURE OF THE INVENTION

In certain embodiments, the present invention provides means and methods for large-scale uses, although they are also of value when applied at smaller scales.

Binding peptides are often used to isolate a particular compound from its environment. The particular binding properties of the binding molecule are typically suited for obtaining reasonably pure preparations of the particular compound. However, when scaling up the technology, it was found that the purity of the particular compound is typically less than in small-scale preparations. There are probably many factors contributing to the reduced purities obtained. Some of these factors include reduced control over the environment containing the particular compound and reduced control over the status of the apparatus used in the separation process and decay. This is particularly true when separation means are being reused for economical reasons.

In the present invention, it was found that the purity of the particular compound is improved significantly when the binding peptide is adapted to the specific environment in which it is intended to perform its binding activity. To this end, the invention in one aspect provides a method for, at least in part, isolating a particular compound from its environment comprising selecting a proteinaceous binding molecule with a binding specificity for the compound and modifying the proteinaceous molecule such that the pKi of the proteinaceous molecule in an aqueous medium is altered when compared to the pKi of the original proteinaceous binding molecule, modification resulting in a reduction of the binding of an undesired compound from the environment to the thus altered proteinaceous binding molecule, the method further comprising providing the altered proteinaceous molecule to the environment to allow binding of the particular compound and separating the altered proteinaceous molecule from the environment. Subsequently, one may further separate the compound from the altered proteinaceous binding molecule and collect the thus isolated compound. The compound may be subjected to further processing or purification steps. However, it is also within the scope of the present invention to remove a particular compound from an environment, for instance, but not limited to, for masking, recycling or detoxification purposes. Removal should, at least in part, reduce the presence or availability of the particular compound from the environment.

The pKi of the proteinaceous molecule is influenced predominantly by amino acid side chains that are exposed to the exterior of the binding molecule. Adapting the pKi of the proteinaceous molecule to the environment of use is preferably done by adapting the pKi of the proteinaceous binding molecule, such that it has an overall charge identical to the major compounds in the environment of use or, preferably, an overall neutral charge. Such adaptation results in improved purity of the particular compound. The adapted pKi also allows improved performance when the means for separating the particular compound are regenerated and reused for another run. Also, after reruns, preparations of a particular compound are purer and have, in general, a higher yield compared to reruns with a proteinaceous binding molecule that is not adapted for the pKi of the environment. Even purer products can be obtained after serial purifications in which in each serial mode, a proteinaceous binding molecule is used that differs from the other proteinaceous binding molecules by means of charge.

The environment of use is preferably the mixture of compounds from which the particular compound needs to be separated. This can be any environment. Preferred environments in the present invention are biological products. Preferred biological products are milk and its derivatives, chemically engineered products, such as drugs, synthetic hormones, antibiotics, peptides, nucleic acids, food additives, etc., plant product streams, such as those obtained from tomato and potato, viruses, blood and its derivatives, secreted products or products stored in cell compartments by micro-organisms, pro- or eukaryotic cells.

Adaptation of the proteinaceous binding molecule can be performed in various ways. It is possible to chemically modify the proteinaceous binding molecule via chemical modification of amino acids with exposure to the exterior of the proteinaceous binding molecule. Such modification typically occurs at reactive amino or carboxyl groups, thereby affecting the pKi of the proteinaceous binding molecule. Chemical or other modifications have the drawback that the result of the modification may vary from batch to batch. Thus, in a preferred embodiment, the proteinaceous binding molecule is altered through amino acid substitution. In this way, a constant property is provided, thereby improving the overall reliability and predictability of subsequent steps. The modification may be in the binding peptide (the compound binding part) of the proteinaceous binding molecule. Considering that changes in the binding peptide very often affect the binding strength and specificity of the proteinaceous binding molecule, it is preferred that the modification is not in the compound binding part of the proteinaceous molecule.

Any type of compound capable of being specifically bound by a proteinaceous binding molecule is suited for the method of the invention. Such compounds typically include proteinaceous molecules, carbohydrates, lipids, nucleic acids, hormones, heavy metals, pesticides, herbicides, antibiotics, drugs, organic compounds, chemically engineered compounds, vitamins, toxins, and chiral compounds. In a preferred embodiment, the compound comprises a proteinaceous molecule. A proteinaceous molecule is a molecule comprising at least two amino acids in peptidic linkage with each other. The proteinaceous molecule is preferably a molecule that is produced by a biological organism or a part, derivative and/or analogue thereof. Thus, parts generated through splicing of a molecule that is produced by a biological organism is also a preferred compound of the invention. It is clear that derivatives generated through modification of the compound after the production by a biological molecule is also a preferred compound of the invention. In a particularly preferred embodiment, the compound comprises antibodies, peroxidases, lactoferrin, growth factors, or coagulation factors.

A proteinaceous binding molecule is a proteinaceous molecule capable of specifically binding a particular compound. This specific binding is, for instance, typical for immunoglobulins. For the present invention, binding is said to be specific if a major compound that is retrieved using proteinaceous binding molecules from complex mixtures containing the compound is the target compound. Affinity for the particular compound is usually in the micromolar range or even lower, whereas the binding affinity for a large number of other compounds is usually in the millimolar range. Thus, it may be clear that with specific binding, it is not excluded that the proteinaceous binding molecule is capable of binding more than one compound with an affinity in the micromolar range or better as long as there is a plurality of compounds that is bound with an affinity in one millimolar range or worse. The binding affinities of specific and non-specific binders should be in the same class of compounds. For instance, if the selective binding compound is a proteinaceous molecule, the non-selective binding molecules are preferably also proteinaceous molecules. In a preferred embodiment of the invention, the proteinaceous binding molecule comprises an immunoglobulin or a functional part, derivative and/or analogue thereof. A functional part, derivative and/or analogue of an immunoglobulin comprises the same compound binding activity in kind, but not necessarily in amount. Examples of such parts, derivatives and/or analogues are Fab fragments, single chain antibody fragments, and the like.

### BRIEF DESCRIPTION OF THE DRAWINGS

FIG. 1: CM126 vector map.

FIG. 2: CM126 sequence (SEQ ID NO: 28).

FIG. 3: iMab100 DNA sequence (SEQ ID NO: 29).

FIG. 4: iMab100 protein sequence (SEQ ID NO: 30).

FIG. 5: Purification of lysozyme from dissolved milk powder (ELK). (1) Molecular weight marker; (2) input (clarified ELK+lysozyme); (3) flow-through (unbound ELK proteins); (4) wash-out (non-specifically bound proteins to Ni-NTA resin); (5) eluate (specifically bound lysozyme).

FIG. 6: Purification of lysozyme from chicken egg white. (1) Chicken egg white (input); (2) flow-through (unbound chicken egg proteins); (3) wash-out (non-specifically bound proteins); (4) eluate (specifically bound lysozyme).

FIG. 7: CM114 vector map.

FIG. 8: CM114-iMab113 DNA sequence (SEQ ID NO: 31).

FIG. 9: CM114-iMab114 DNA sequence (SEQ ID NO: 32).

FIG. 10: Protein sequences for VAPs with bovine LF-binding characteristics. iMab142-02-0002 (SEQ ID NO: 33), iMab142-02-0010 (SEQ ID NO: 34) and iMab142-02-0011 (SEQ ID NO: 35) code for nine-stranded VAPs while iMab143-02-0012 (SEQ ID NO: 36), iMab143-02-0013 (SEQ ID NO: 37) and iMab144-02-0014 (SEQ ID NO: 38) code for seven-stranded VAPs. The scaffolds of iMab142 series are identical. The scaffolds of iMab143 series are also identical. All VAPs have affinity for bovine lactoferrin proteins. The affinity region 4 is indicated in bold. The affinity region 4 of two of the selected binders, the nine-stranded iMab142-02-0011 and the seven-stranded iMab143-02-0012, is identical.

FIG. 11: Binding of lactoferrin to iMab142-02-0002 and iMab142-02-0010. The iMabs were immobilized on the column as in Example 10 and 18 ml of 0.2 mg/ml lactoferrin was loaded on the column. After loading, the column is washed with 5 volumes of PBS pH 7+20 mM imidazole to remove non-specifically bound proteins. After washing, the specific bound LF was eluted with 2 ml 1 M NaCl (elution 1) and 2 ml 2 M NaCl (elution 2), respectively. Fractions of all steps were collected and analyzed on SDS-PAGE. Lane 1, Flow-through; Lane 2, Elution 1; Lane 3, Elution 2; Lane 4, Lactoferrin 0.2 mg/ml; Lane 5, Marker; Lane 6, Flow-through; Lane 7, Wash; Lane 8, Elution 1; Lane 9, Elution 2.

FIG. 12: Purification of lactoferrin from casein whey. (1) input (clarified casein whey); (2) flow-through (unbound whey proteins); (3) eluate (specifically bound lactoferrin).

FIG. 13: iMab1300 DNA sequence (SEQ ID NO: 39).

FIG. 14: iMab1500 DNA sequence (SEQ ID NO: 40).

FIG. 15: DNA sequence and adapted restriction site of iMab143-02-0003 (SEQ ID NO: 41) and iMab144-02-0003 (SEQ ID NO: 42). DNA sequence of coding region of iMab143-02-0003 (SEQ ID NO: 41) and iMab144-02-0003 (SEQ ID NO: 42). The adapted restriction sites HindIII, EcoRI and PstI for around affinity region 4 are indicated in bold. The open reading frames code for seven-stranded iMabs including affinity regions for lysozyme. Affinity region 4 serves as a dummy region for library construction.

FIG. 16: Binding of lactoferrin to 144-02-0011, iMab143-02-001 and iMab143-02-0013. The iMabs were immobilized on the column and 18 ml of 0.2 mg/ml lactoferrin was loaded on the column. After loading, the column is washed with 5 volumes of PBS pH 7+20 mM imidazole to remove non-specifically bound proteins. After washing, the specific bound LF was eluted with 2 M NaCl in portions of two (elution 1) and one (elution 2) ml, respectively. Fractions of all steps were collected and analyzed on SDS-PAGE. Lane 1, Lactoferrin 0.2 mg/ml; Lane 2, Flow-through; Lane 3, Wash; Lane 4, Elution 1; Lane 5, Elution 2; Lane 6, Flow-through; Lane 7, Wash; Lane 8, Elution 1; Lane 9, Elution 2; Lane 10, Marker; Lane 11, Flow-through; Lane 12, Wash; Lane 13, Elution 1; Lane 14, Elution 2.

FIG. 17: Schematic 3D-topology of scaffold domains. Eight example topologies of protein structures that can be used for the presentation of antigen binding sites are depicted. The basic core beta elements are denominated in Example A. This basic structure contains nine beta-elements positioned in two plates. One beta-sheet contains elements 1, 2, 6 and 7 and the other contains elements 3, 4, 5, 8 and 9. The loops that connect the beta-elements are also depicted. Bold lines are connecting loops between beta-elements that are in top position while dashed lines indicate connecting loops that are located in bottom position. A connection that starts dashed and ends solid indicates a connection between a bottom and top part of beta-elements. The numbers of the beta-elements depicted in the diagram correspond to the numbers and positions mentioned in FIGS. 1 and 2. Panel A: nine-beta-element topology, for example, all antibody light and heavy chain variable domains and T-cell receptor variable domains; Panel B: eight-beta-element topology, for example, interleukin-4 alpha receptor (1IAR); Panel C: seven-beta-element topology, for example, immunoglobulin killer receptor 2dl2 (2DLI); Panel D: seven-beta-element topology, for example, E-cadherin domain (1FF5); Panel E: six-beta-strand topology; Panel F: six-beta-element topology, for example, Fc epsilon receptor type alpha (1J88); Panel G: six-beta-element topology, for example, interleukin-1 receptor type-1 (1GOY); and Panel H: five-beta-element topology.

### DETAILED DESCRIPTION OF THE INVENTION

In a particularly preferred embodiment, a proteinaceous binding molecule of the invention comprises a synthetic or recombinant proteinaceous molecule comprising a binding peptide and a core, the core comprising a β-barrel comprising at least four strands, wherein the β-barrel comprises at least two β-sheets, wherein each of the β-sheets comprises two of the strands and wherein the binding peptide is a peptide connecting two strands in the β-barrel and wherein the binding peptide is outside its natural context. Preferably, a synthetic or recombinant proteinaceous molecule comprising a binding peptide and a core, the core comprising a β-barrel comprising at least five strands, wherein the β-barrel comprises at least two β-sheets, wherein at least one of the β-sheets comprises three of the strands and wherein the binding peptide is a peptide connecting two strands in the β-barrel and wherein the binding peptide is outside its natural context. This core structure has been identified in many proteins, ranging from galactosidase to human (and, e.g., camel) antibodies with all kinds of molecules in between. Nature has apparently designed this structural element for presenting desired peptide sequences. This core has now been produced in an isolated form, as well as many variants thereof that still have the same or similar structural elements. These novel structures can be used in all applications where other binding molecules are used and even beyond those applications as explained herein. The structure comprising one affinity region (desired peptide sequence or binding peptide) and two β-sheets forming one β-barrel is the most basic form of the invented proteinaceous binding molecules. “Proteinaceous” means that they are, in essence, amino acid sequences, but that side chains and/or groups of all kinds may be present; it is, of course, possible, since the amino acid sequence is of less relevance for the structure, to design other molecules of a non-proteinaceous nature that have the same orientation in space and can present peptidic affinity regions; the orientation in space is the important parameter.

The invention also discloses optimized core structures in which less stable amino acids are replaced by more stable residues (or vice versa) according to the desired purpose. Of course, other substitutions or even amino acid sequences completely unrelated to existing structures are included since, once again, the important parameter is the orientation of the molecule in space.

In certain embodiments of the invention, it is preferred to apply a more advanced core structure than the basic structure, because both binding properties and structural properties can be designed better and with more predictive value. Thus, the invention preferably provides a proteinaceous molecule according to the invention wherein the β-barrel comprises at least five strands, wherein at least one of the sheets comprises three of the strands, more preferably a proteinaceous molecule according to the invention, wherein the β-barrel comprises at least six strands, wherein at least two of the sheets comprises three of the strands. β-barrels, wherein each of the sheets comprises at least three strands, are sufficiently stable while, at the same time, providing sufficient variation possibilities to adapt the core/affinity region (binding peptide) to particular purposes, though suitable characteristics can also be found with cores that comprise less strands per sheet. Thus, variations wherein one sheet comprises only two strands are within the scope of the present invention.

In an alternative embodiment, the invention provides a proteinaceous molecule according to the invention wherein the β-barrel comprises at least seven strands, wherein at least one of the sheets comprises four of the strands. Alternatively, the invention provides a proteinaceous molecule according to the invention, wherein the β-barrel comprises at least eight strands, wherein at least one of the sheets comprises four of the strands.

In another embodiment, a proteinaceous molecule according to the invention, wherein the β-barrel comprises at least nine strands, wherein at least one of the sheets comprises four of the strands, is provided. In the core structure, there is a more open side where nature displays affinity regions and a more closed side, where connecting sequences are present. Preferably, at least one affinity region is located at the more open side.

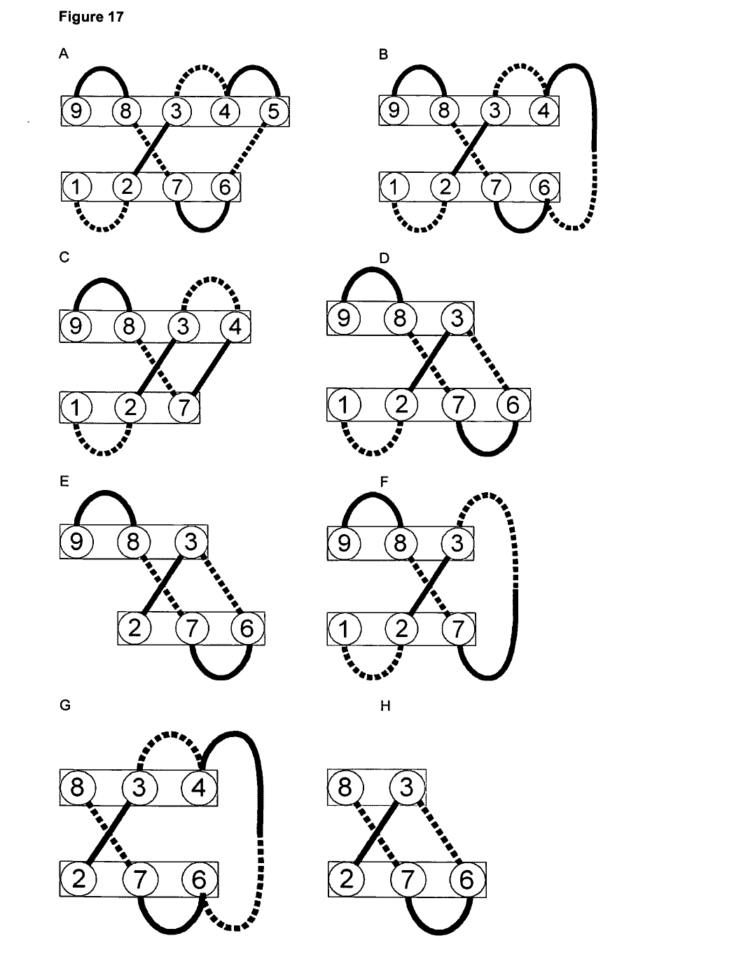

Thus, the invention provides a proteinaceous molecule according to the invention, wherein the binding peptide connects two strands of the β-barrel on the open side of the barrel. Although the location of the desired peptide sequence (affinity region) may be anywhere between two strands, it is preferred that the desired peptide sequence connects the two sheets of the barrel. Thus, the invention provides a proteinaceous molecule according to the invention, wherein the binding peptide connects at least two β-sheets of the barrel. Although one affinity region may suffice, it is preferred that more affinity regions are present to arrive at a better binding molecule. Preferably, these regions are arranged such that they can cooperate in binding (e.g., both on the open side of the barrel). Thus, the invention provides a proteinaceous molecule according to the invention, which comprises at least one further binding peptide. A successful element in nature is the one having three affinity regions and three connecting regions. This element in its isolated form is a preferred embodiment of the present invention. However, because of the versatility of the presently invented binding molecules, the connecting sequences on the less open side of the barrel can be used as affinity regions as well. This way, a very small bispecific binding molecule is obtained. Thus, the invention provides a proteinaceous molecule according to the invention, which comprises at least four binding peptides. “Bispecific” herein means that the binding molecule has the possibility to bind to two target molecules (the same or different). The various strands in the core are preferably encoded by a single open reading frame. The loops connecting the various strands may have any type of configuration. So as not to unduly limit the versatility of the core, it is preferred that loops connect strands on the same side of the core, i.e., an N-terminus of strand (a) connects to a C-terminus of strand (b) on either the closed side or the open side of the core. Loops may connect strands in the same β-sheet or cross-over to the opposing β-sheet. A preferred arrangement for connecting the various strands in the core is given in the examples and the figures and, in particular, FIG. 17. Strands in the core may be in any orientation (parallel or antiparallel) with respect to each other. Preferably, the strands are in the configuration as depicted in FIG. 17. Molecules of this kind are referred to herein as VAPs. VAPs and their generation and uses are further detailed in PCT/NL02/00810 and EP 01204762.7, which are incorporated by reference herein.

With the “pKi of the proteinaceous binding molecule” is meant the pH in aqueous solution at which the net charge of the proteinaceous binding molecule is neutral. In the present invention, the pKi is preferably adapted such that the proteinaceous binding molecule has no noticeable net charge in the environment of use. This can be at least approximated by allowing for a variation of the pKi of the adapted proteinaceous binding molecule and the pH of the environment of use. This variation preferably comprises less than pH 1.0, preferably less than pH 0.50 and, particularly preferred, less than pH 0.25.

The present invention also provides the altered proteinaceous binding molecule that is used in a method of the invention described above. This proteinaceous binding molecule comprises a binding peptide and a core for at least partly isolating a particular compound from its environment wherein the proteinaceous molecule is adapted for improved binding specificity of the compound in the environment. This improved binding specificity is preferably the result of an altered pKi as compared to the original proteinaceous binding molecule. However, other adaptations are also provided.

In a preferred embodiment, the adaptation comprises the addition or removal of an amino acid exposed to the exterior of the proteinaceous binding molecule, wherein the amino acid is capable of chemical linkage with a carrier surface. It is often practical to couple the proteinaceous binding molecule to a solid surface. This at least allows easy separation of the bound compound from the environment. The coupling to a solid surface can be performed in various ways. In a preferred embodiment, the coupling is performed through chemical linkage of reactive amino acid exposed to the exterior of the proteinaceous binding molecule. Preferred reactive groups are reactive amino or carboxyl groups in the amino acid side chain. In a preferred embodiment, the reactive amino acid is a glycine. In one aspect of the invention, the addition or removal of the amino acid exposed to the exterior is used to tailor the orientation of the proteinaceous binding molecule on the solid surface. By adding or removing reactive amino acids, it is possible to create or delete coupling sites in the proteinaceous binding molecule and thereby direct the orientation of the proteinaceous binding molecule on the solid surface. Addition or removal may be within the amino acid chain or at the ends. Optimizing the orientation also improves the specific compound binding properties of the proteinaceous binding molecule on the solid surface and thereby in the environment. Optimizing the orientation also allows a decrease in the non-specific binding of undesired compounds in the environment. Both effects lead to increased purity of the separated product. Washing conditions also affect the purity of the compound after separation. When in the present invention reference is made to “increased purity,” this is referred to in the situation of extensive washing. The term “washing” herein refers to the normal activity in the field of protein purification wherein, for instance, affinity columns are washed with solution to remove unbound compounds and compounds that are bound non-specifically. Thus, the increased purity that is possible of being obtained by the present invention can be traded with reduced washing, thereby also simplifying the process. In large-scale applications, even reduced washing conditions can mean serious economic advantage. The present invention not only provides adaptation of the proteinaceous binding molecule to the environment of use, but also the adaptation of the proteinaceous binding molecule bound to the solid surface for use in the environment.

Specific VAPs can be used to remove target molecules from complex fluids. Retrieval of such target molecules can be done in basically two ways: via direct capturing and via indirect capturing. Direct capturing requires VAPs that have been immobilized on a carrier material. This way, target molecules are captured from solutions and kept on the surface of the carrier material until elution. In indirect capturing methods, VAPs are added to the solutions that contain target molecules. After hybrid formation (VAP-target), the fluid is brought in contact with a matrix that is able to bind the VAP. Binding of VAP-target hybrids can be accomplished using specific affinity-tags or regions including poly-His, FLAG-tag, Strep-tag, or other specific adaptations or via the use of binding molecules that specifically recognize the VAP structure, e.g., VAP1 against VAP2.

Immobilization of iMab molecules on carrier material should preferably be accomplished in a unidirectional fashion, i.e., with the affinity regions of the iMab proteins remote from the carrier surface. This way, target molecules can be captured with maximum efficiency and maximum load capacity. There are several means to position proteins onto surface materials. One way to immobilize proteins onto a carrier surface is via the use of a chemical reaction between amino acid side chains and reactive groups on the surface of the carrier material. A preferred amino acid side chain that can accomplish such a reaction with epoxy-groups is, for example, the reactive free amino group present in the amino acid lysine. The free amino group present in lysine can react under relative mild conditions with epoxy groups resulting in a covalent bond. Proteins that contain lysine residues at aberrant positions can be immobilized onto epoxy-activated resins with reduced or even completely lacking target capturing properties. Maximum binding efficiency of ligands on iMab-loaded resins can be accomplished by the removal or displacement of lysine residues in such a way that aberrant positioning of the iMabs cannot, or at low percentages, occur. Removal of aberrant lysine residues can be done by several means, among which computer modeling-mediated amino acid replacements. Lysine residues can be inserted at desired locations or positions in the proteins as predicted again with computer-aided modeling or by the addition of a lysine-containing tail region, e.g., at the carboxy terminal of iMab molecules.

Besides reactive amino group immobilization strategies, carboxy, hydroxyl or other amino acid side chains can be used. Reversible immobilizations can also be applied. Such interaction can be found between 6*his-tails and Ni-beads or other weak interactive tags (Strep-tag, GST, Flag-tag, etc).

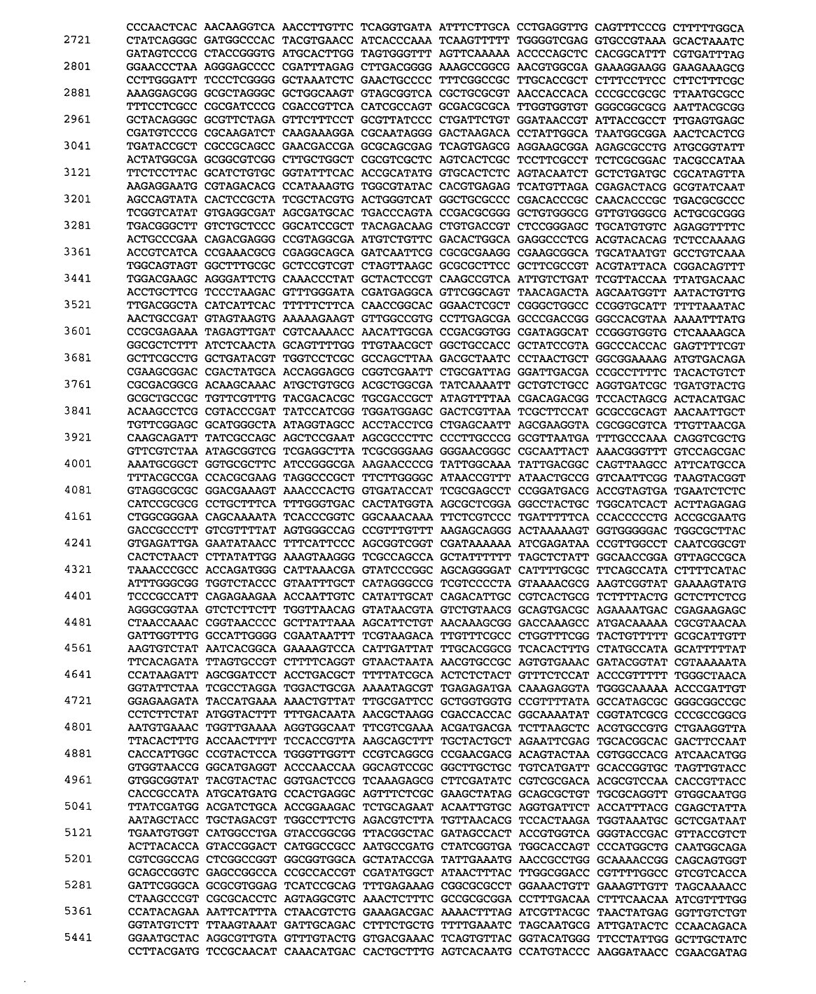

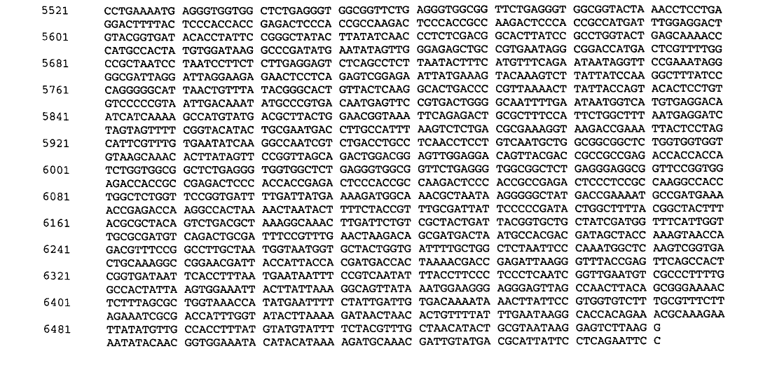

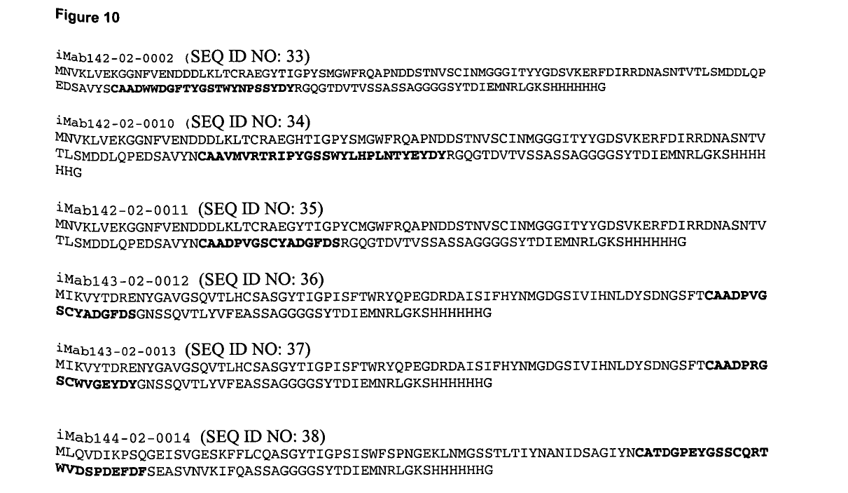

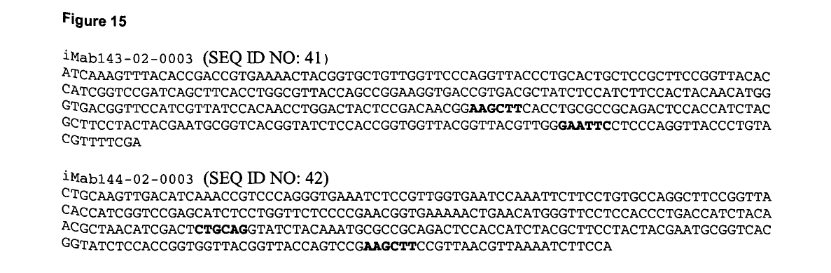

In one aspect, the invention provides proteinaceous binding molecules comprising a sequence or encoded by a sequence as depicted in FIGS. 10 or 15 or Table 1 (except iMab100). The proteinaceous binding molecules may, of course, also be provided in a functional part as long as the binding specificity of the part is the same in kind, not necessarily in amount, as the depicted proteinaceous binding molecule. Also provided are functional derivatives of the depicted proteinaceous binding molecule, wherein the derivative is a chemical or biological modification of the proteinaceous binding molecule. Functional analogues are also provided. It is possible to generate the same binding specificity in kind, not necessarily in amount, as a proteinaceous binding molecule depicted in the figure by, for instance, protein mimics. Such mimics are also part of the invention. Also provided is a nucleic acid encoding a proteinaceous binding molecule comprising a sequence or encoded by a sequence depicted in FIGS. 10 or 15 or Table 1 (except iMab100) or a cell comprising such a nucleic acid. Such cells may be used for the production of the proteinaceous binding molecule. In one embodiment, such a cell is a prokaryotic cell. Considering the wide availability of cores and suitable binding peptides, it is possible to graft a different binding peptide onto a core or vice versa. Thus, the invention further provides the various interchanges of cores and binding peptides of the proteinaceous binding molecules depicted in FIGS. 10 or 15 or Table 1 (except iMab100). The invention further provides a proteinaceous binding molecule of the invention provided with a different specific binding peptide, either in addition to or in place of the first binding peptide. Since both the cores and the binding peptides may be used in such MASTing, the invention also provides a proteinaceous binding molecule wherein at least part of the binding peptide is removed. On the other side, the invention also provides a binding peptide comprising a sequence or encoded by a sequence depicted in FIGS. 10 or 15 or Table 1 (except iMab100) wherein at least part of the core is removed.

In yet another aspect, the invention provides a proteinaceous binding molecule comprising a binding specificity for a lactoferrin form, a lactoperoxidase, a growth factor, an antibody, a lysozyme, or an oligosaccharide. Preferably, wherein the proteinaceous binding molecule comprising a synthetic or recombinant proteinaceous molecule comprising a binding peptide and a core, the core comprising a β-barrel comprising at least four strands, wherein the β-barrel comprises at least two β-sheets, wherein each of the β-sheets comprises two of the strands and wherein the binding peptide is a peptide connecting two strands in β-barrel and wherein the binding peptide is outside its natural context.

The term “core” is used to relate to a VAP without affinity loops that can have one or more connecting loops. When explicitly VAP without affinity but with connecting loops is related to, the term “scaffold” is used.

The invention is further explained with the aid of the following illustrative Examples.

### EXAMPLES

### Example 1

### Changing Amino Acids in the Exterior: Changing pI Values

Amino acids on the exposed side (exterior) of proteins determine putative interactions with other molecules. Especially charged amino acids like basic (lysine, histidine and arginine) and acidic residues (aspartic acid and glutamic acid) can charge proteins under certain pH conditions. Highly charged proteins can easily stick to other molecules that have opposite charges. This sticky property is not always desired, especially not when the interaction takes place at regions that are not involved in specific binding. All proteins have an iso-electric point at which the charge of the protein as a whole is neutrally charged. At the pI, aspecific charge-based interactions are assumed to be minimal. Each industrial application makes use of fluid streams that can differ in pH to a large extent. This means that in some applications the pI of the VAP should be different than a VAP used in other processes. Therefore, several VAPs were engineered, each of this with its unique pI.

Inspection of the iMab100 structure and sequence showed that several putatively charged residues are located on the surface of the fold. These residues can be exchanged with other amino acids resulting in proteins with different pI values and thus with different interacting properties. Template- or homology-modeling strategies with Modeller software were applied for these residues. The reliability of each new amino acid exchange was assessed with Prosall, What-if and Procheck. Some of the new models contained amino acid replacements that were unfavorable because of the chemical or physical nature of these exchanged amino acids. Cysteine, for example, could make the proteins susceptible to covalent dimerization with proteins that also bear a free cysteine group. The introduction of ATG sequences might result in alternative protein products due to potential alternative start sites. Methionine residues were only assessed if no other amino acids could give satisfactory results. All other amino acid residues were assessed with ProsaII, What-if and Procheck. Proposed replacements for the charged residues were indicated to yield valid models (Table 1). After modeling, both theoretical and practical pI values were determined. Theoretical values were generated using the program “Gene Runner” from Hastings Software Incorporated (version 3.02; Tables 2 and 3). Practical pI was determined with iso-electric focusing using standardized procedures as indicated by the manufacturers (Table 2).

### Example 2

### Assembly of Synthetic Scaffolds

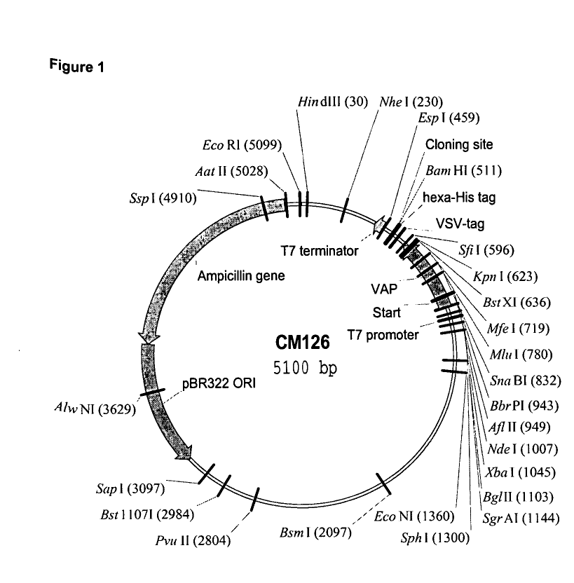

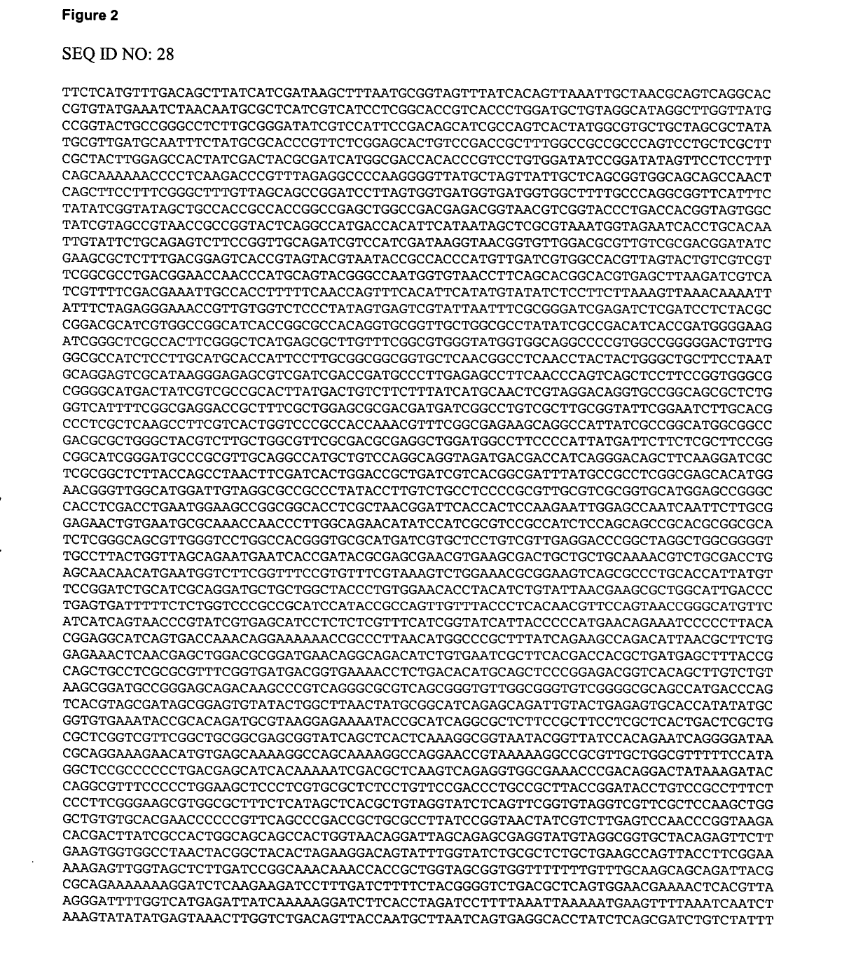

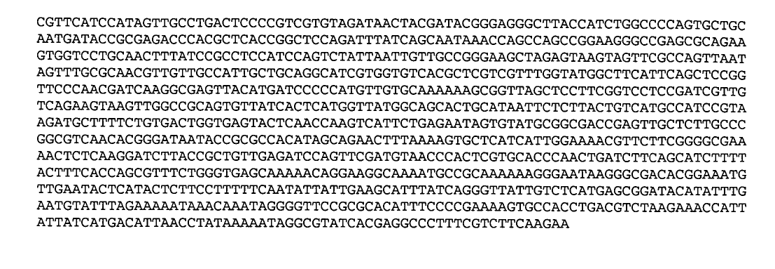

Synthetic VAPs were designed on the basis of their predicted three-dimensional structure. The amino acid sequence was back translated into DNA sequence using the preferred codon usage for enteric bacterial gene expression. The obtained DNA sequence was checked for undesired restriction sites that could interfere with future cloning steps. Such sites were removed by changing the DNA sequence without changing the amino acid codons. Next, restriction site overhanging regions were added enabling unidirectional cloning of the DNA sequence. PCR assembly consists of four steps: oligo primer design (ordered at Operon's), gene assembly, gene amplification, and cloning. The scaffolds were assembled in the following manner: first, both plus and minus strands of the DNA sequence were divided into oligonucleotide primers of approximately 35 bp and the oligonucleotide primer pairs that code for opposite strands were designed in such a way that they have complementary overlaps of approximately 16 to 17 bases. Second, all oligonucleotide primers for each synthetic scaffold were mixed in equimolar amounts, 100 pmol of this primer mix was used in a PCR assembly reaction using 1 Unit Taq polymerase (Roche), 1×PCR buffer+mgCl₂ (Roche) and 0.1 mM dNTP (Larova) in a final volume of 50 μl, 35 cycles of 30 seconds at 92° C., 30 seconds at 50° C., and 30 seconds at 72° C. Third, 5 μl of PCR assembly product was used in a standard PCR amplification reaction using, both outside primers of the synthetic scaffold, 1 Unit Taq polymerase, 1×PCR buffer+MgCl₂, and 0.1 mM dNTP in a final volume of 50 μl, 25 cycles of 30 seconds at 92° C., 30 seconds at 55° C., and 1 minute at 72° C. Fourth, PCR products were analyzed by agarose gel electrophoresis. PCR products of the correct size were digested with correct restriction enzymes and ligated into vector CM126 (FIGS. 1 and 2) linearized with the same restriction enzyme set as used for the digestion of the synthesized fragments. Ligation products were transformed into competent bacterial cells like TOP10 (InVitrogen), E.cloni (Lucigen), TG1 (Stratagene), X11-blue (Stratagene) or other convenient cells, and grown overnight at 37° C. on 2×TY plates containing corresponding antibiotics and 2% glucose. Single colonies were grown in liquid medium containing corresponding antibiotics and plasmid DNA was isolated (Promega) and used for sequence analysis (Beckmann Coulter Seq8000).

### Example 3

### Expression Vector CM126 Construction

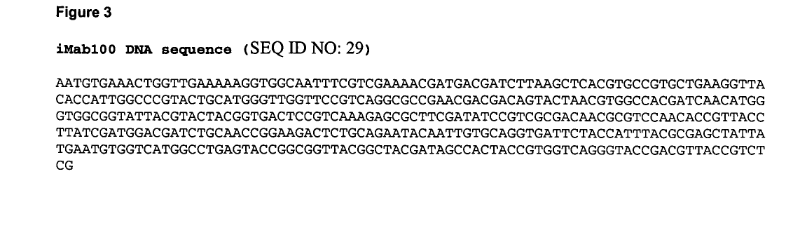

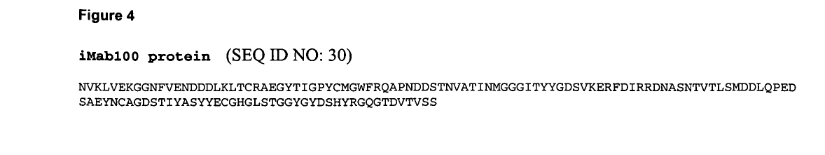

A vector for efficient protein expression (CM126; see FIGS. 1 and 2) based on pET-12a (Novagen) was constructed. A dummy VAP, iMab100 (FIGS. 3 and 4), including convenient restriction sites, linker, VSV-tag, six times His-tag and stop codon was inserted. As a result, the signal peptide OmpT was omitted from pET-12a. iMab100 was PCR amplified using a forward primer that contains a 5′ NdeI overhanging sequence and a very long reverse oligonucleotide, a reverse primer containing all linkers and tag sequences and a BamHI overhanging sequence. After amplification, the PCR product and pET-12a were digested with NdeI and BamHI. After gel purification, products were purified via the Promega gel-isolation system according to the manufacturer's procedures. The vector and PCR fragment were ligated and transformed by electroporation in E. coli TOP10 cells. Correct clones were selected and verified for their sequence by sequencing. This vector including the dummy VAP acted as the basic vector for expression analysis of other VAPs. Insertion of other VAPs was performed by amplification with specific primers, digestion with corresponding restriction enzymes and ligation into digested CM126.

### Example 4

### Expression of VAPs

E. coli BL21 (DE3) (Novagen) was transformed with expression vector CM126-VAP. Cells were grown in 250 ml shaker flasks containing 50 ml 2*TYmedium (16 g/l tryptone, 10 g/l yeast extract, 5 g/l NaCl (Merck)) supplemented with ampicillin (200 microgram/milliliter) and agitated at 30° C. Isopropylthio-β-galactoside (IPTG) was added at a final concentration of 0.2 mM to initiate protein expression when OD (600 nm) reached one. The cells were harvested four hours after the addition of IPTG, centrifuged. (4000 g, 15 minutes, 4° C.) and pellets were stored at −20° C. until used. Protein expression was analyzed by Sodium Dodecyl Sulphate PolyAcrylamide Gel Electrophoresis (SDS-PAGE).

### Example 5

### Purification of VAPs from Inclusion Bodies Using Heat

VAP proteins are expressed in E. Coli BL21 (CM126-iMab100) as described in Example 4. Inclusion bodies are purified as follows. Cell pellets (from a 50 ml culture) are resuspended in 5 ml PBS pH 8 up to 20 g cdw/l and lysed by two passages through a cold French pressure cell (Sim-Aminco). Inclusion bodies are collected by centrifugation (12,000 g, 15 minutes) and resuspended in PBS containing 1% Tween-20 (ICN) in order to solubilize and remove membrane-bound proteins. After centrifugation (12,000 g, 15 minutes), pellet (containing inclusion bodies) is washed two times with PBS. The isolated inclusion bodies are resuspended in PBS pH 8+1% Tween-20 and incubated at 60° C. for 10 minutes. This results in nearly complete solubilization of most VAPs. The supernatant is loaded on a Nickel-Nitrilotriacetic acid (Ni-NTA) superflow column and purified according to a standard protocol as described by Qiagen (The QIAexpressionist™, fifth edition, 2001). The binding of the purified VAPs is analyzed by ELISA techniques.

### Example 6

### Purification of VAPs Proteins from Inclusion Bodies Using Urea and Matrix-Assisted Refolding

Alternatively, VAPs are solubilized from inclusion bodies using 8 m urea and purified into an active form by matrix-assisted refolding. Inclusion bodies are prepared as described in Example 5 and solubilized in 1 ml PBS pH 8+8 m urea. The solubilized proteins are clarified from insoluble material by centrifugation (12,000 g, 30 minutes) and subsequently loaded on a Ni-NTA superflow column (Qiagen) equilibrated with PBS pH 8+8 M urea. A specific proteins are released by washing the column with 4 volumes PBS pH 6.2+8 M urea. The bound His-tagged VAP proteins are allowed to refold on the column by a stepwise reduction of the urea concentration in PBS pH 8 at room temperature. The column is washed with 2 volumes of PBS+4 M urea, followed by 2 volumes of PBS+2 M urea, 2 volumes of PBS+1 M urea and 2 volumes of PBS without urea. VAP proteins are eluted with PBS pH 8 containing 250 mM imidazole. The released VAP proteins are dialyzed overnight against PBS pH 8 (4° C.), concentrated by freeze drying and characterized for binding and structure measurements. The purified fraction of VAP proteins are analyzed by SDS-PAGE.

### Example 7

### Purification of Lysozyme from Dissolved Milk Powder (ELK) Using Affinity Chromatography

Lysozyme from dissolved milk powder will be purified using direct affinity chromatography. IMab molecules with specific affinity against lysozyme are immobilized to nickel-nitrilo acetic acid (Ni-NTA)-agarose resin and subsequently exposed to dissolved milk powder. After washing, specifically bound lysozyme can be eluted using a NaCl gradient.

Immobilization of iMab100

Purified iMab100 was immobilized by metal affinity chromatography via specific binding of the 6*His affinity tag to nickel-nitrilo acetic acid (Ni-NTA)-agarose resin. IMab100 (100 mg) was mixed with 25 ml Ni-NTA-superflow resin, incubated for one hour in 10 mM phosphate buffer+137 mM NaCl (PBS) pH 8, packed in a column, washed with PBS pH 8+20 mM imidazole to remove a specific bound proteins and subsequently equilibrated with PBS pH 8.

Preparation of Lysozyme in Dissolved Milk Powder

Milk powder (ELK, Campina) was dissolved in 10 mM phosphate buffer (PB) pH 7 up to 0.25% (w/v) and centrifuged (25,000 rpm, one hour) to remove insoluble proteins. Supernatant was further clarified by filtration using a 0.45 μm filter. Lysozyme was mixed with clarified ELK up to a concentration of 10 μg/ml.

Purification of Lysozyme from a Complex Protein Mixture

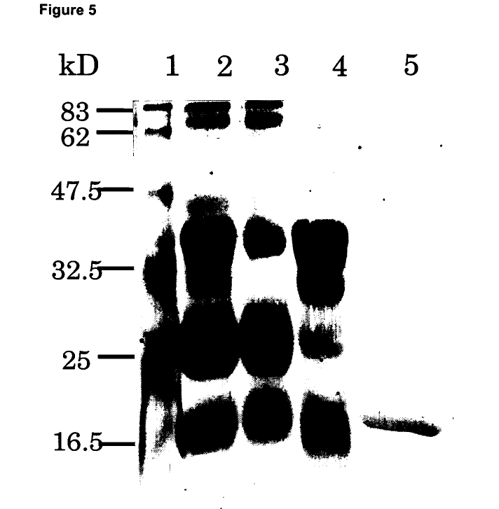

Clarified ELK (150 ml) with lysozyme (10 μg/ml) was loaded on an equilibrated Ni-NTA column immobilized with iMab100. After loading, the column is washed with 5 column volumes of PBS pH 7+25 mM imidazole to remove non-specifically bound proteins. After washing, a linear NaCl gradient (0 to 1 M NaCl in PBS pH 7+25 mM imidazole) is applied to elute specific bound proteins. All steps were performed at a flow rate of 10 ml/minute. Fractions (flow-through, wash-out and eluate) are collected and analyzed using SDS-PAGE and silver staining (FIG. 5).

A single protein is found in the eluate (eluting at 0.4 M NaCl) and corresponds to lysozyme as evidenced by gel filtration using pure chicken egg white lysozyme as reference. The eluate peak was collected manually in fractions of 0.2 ml. The fraction with the highest protein content was measured to be 1.05 mg/ml, showing a 105-fold concentration as compared to the input fraction (10 μg lysozyme/ml).

As a negative control, a Ni-NTA matrix (25 ml) without immobilized iMab100 was used. No protein peaks were found in the eluate, showing that the interaction of lysozyme with iMab100 is specific.

### Example 8

### Purification of Lysozyme from Chicken Egg White by Affinity Chromatography

Lysozyme from chicken egg white can be purified using direct affinity chromatography. IMab molecules with specific affinity against lysozyme can be immobilized to nickel-nitrilo acetic acid (Ni-NTA)-agarose resin and subsequently exposed to chicken egg white. After washing, specifically bound lysozyme can be eluted using a NaCl gradient.

Immobilization of iMab100

Purified iMab100 (with specific affinity towards lysozyme) was immobilized by metal affinity chromatography via specific binding of the 6*His affinity tag to nickel-nitrilo acetic acid (Ni-NTA)-agarose resin. IMab100 (25 mg) was mixed with 25 ml Ni-NTA-superflow resin, incubated for one hour in 10 mM phosphate buffer+137 mM NaCl (PBS) pH 8, packed in a column, washed with PBS pH 8+20 mM imidazole to remove a specific bound proteins and subsequently equilibrated with PBS pH 8.

Preparation of Chicken Egg White

Chicken egg white of a fresh egg was diluted to 150 ml in 10 mM phosphate buffer (PB) pH 7, centrifuged (12,000 rpm, 30 minutes) to remove insoluble and precipitated proteins and subsequently filtered (0.45 μm filter).

Purification of Lysozyme from Chicken Egg White

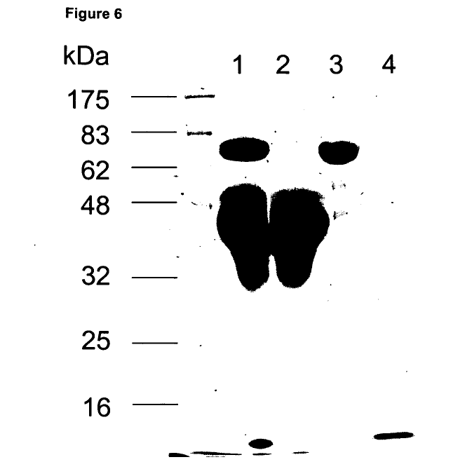

Chicken egg white (50 ml in PBS pH 7) was loaded on an equilibrated Ni-NTA column immobilized with iMab100. After loading, the column is washed with 5 column volumes of PBS pH 7+25 mM imidazole to remove non-specifically bound proteins. After washing, a linear NaCl gradient (0 to 1 M NaCl in PBS pH 7+25 mM imidazole) is applied to elute specific bound proteins. All steps were performed at a flow rate of 10 ml/minute. Fractions (flow-through, wash-out and eluate) are collected and analyzed using SDS-PAGE (FIG. 6).

A single protein is found in the eluate (eluting at 0.4 M NaCl) and corresponds to lysozyme. The protein band is absent in flow-through and wash-out fractions indicating that all lysozyme (mg) could be recovered.

As a negative control, a Ni-NTA matrix (25 ml) without immobilized iMab100 was used. No protein peaks were found in the eluate, showing that the interaction of lysozyme with iMab100 is specific.

### Example 9

### Isolation and Identification of Lactoferrin Binding Nine-Beta-Stranded iMabs

A nucleic acid phage display library having variegations in affinity region 4 (AR4) was prepared by the following method. Llama glama blood lymphocytes were isolated from llamas immunized with lactoferrin according to standard procedures as described in Spinelli et al. (Biochemistry 39 (2000) 1217-1222). RNA from these cells was isolated via Qiagen RNeasy methods according to the manufacturer's protocol. cDNA was generated using muMLv or AMW (New England Biolabs) according to the manufacturer's procedure. CDR3 regions from Vhh cDNA were amplified using 1 μl cDNA reaction in 100 microliters PCR reaction mix comprising 2 units Taq polymerase (Roche), 200 μM of each dNTP (Roche), buffers (Roche Taq buffer system), 2.5 μM of forward and reverse primers in a Primus96 PCR machine (MWG) with the following program 35 times (94° C. for 20 seconds, 50° C. for 25 seconds, 72° C. for 30 seconds). In order to select for CDR3 regions containing at least one cysteine, primer 56 (Table 4) was used as a forward primer and in the case of selecting for CDR regions that do not contain cysteines, primer 76 (Table 4) was used in the first PCR round. In both cases, primer 16 (Table 4) was used as reverse primer. Products were separated on a 1% agarose gel and products of the correct length (˜250 bp) were isolated and purified using Qiagen gel extraction kit. Five μl of these products were used in the next round of PCR, similar to that described above in which primer 8 (Table 4) and primer 9 (Table 4) were used to amplify CDR3 regions. Products were separated on a 2% agarose gel and products of the correct length (80 to 150 bp) were isolated and purified using Qiagen gel extraction kit. In order to adapt the environment of the camelidae CDR3 regions to our scaffold, two extra rounds of PCR similar to the first PCR method were performed on 5 μl of the products with the exception that the cycle number was decreased to 15 cycles and in which primer 73 (Table 4) and 75 (Table 4) were subsequently used as forward primer and primer 49 (Table 4) was used as reverse primer.

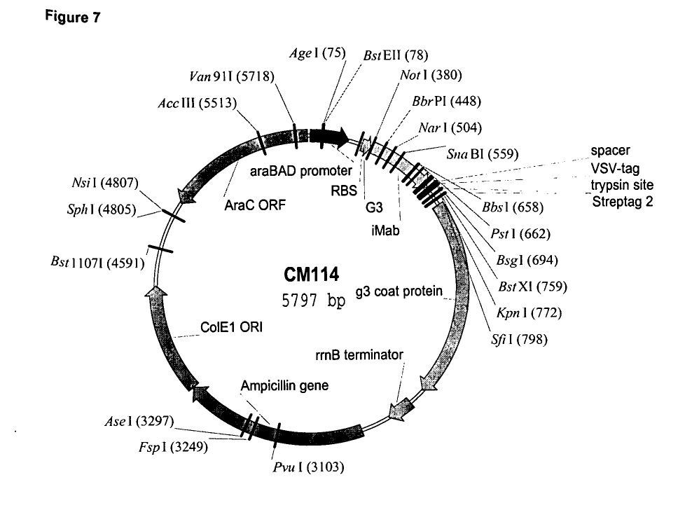

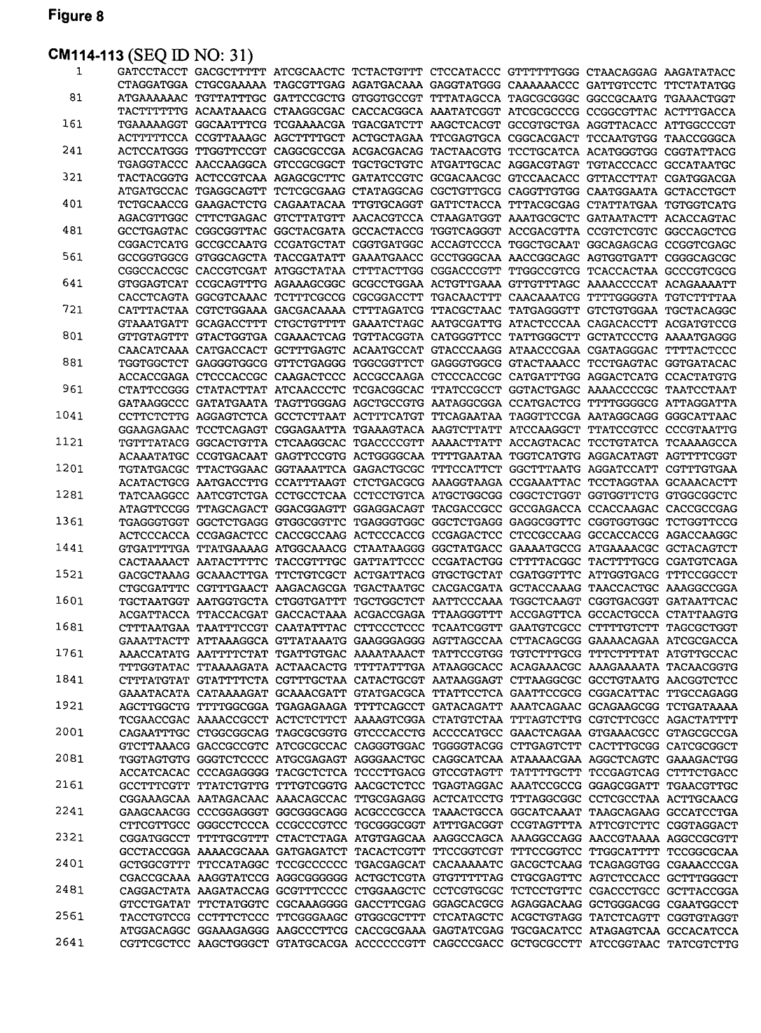

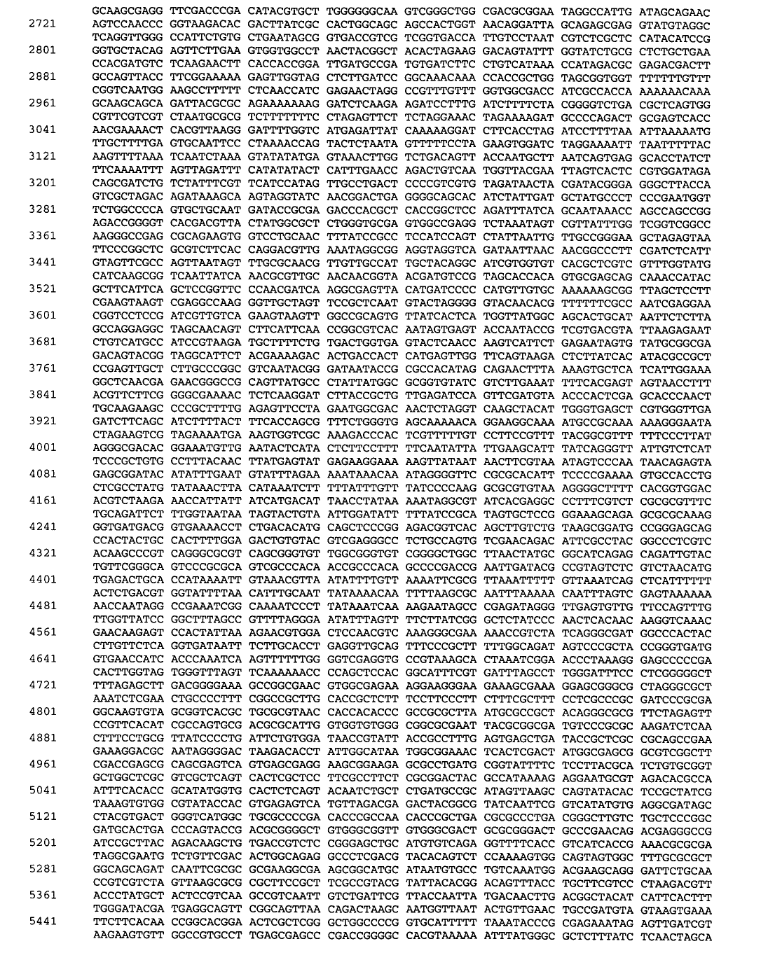

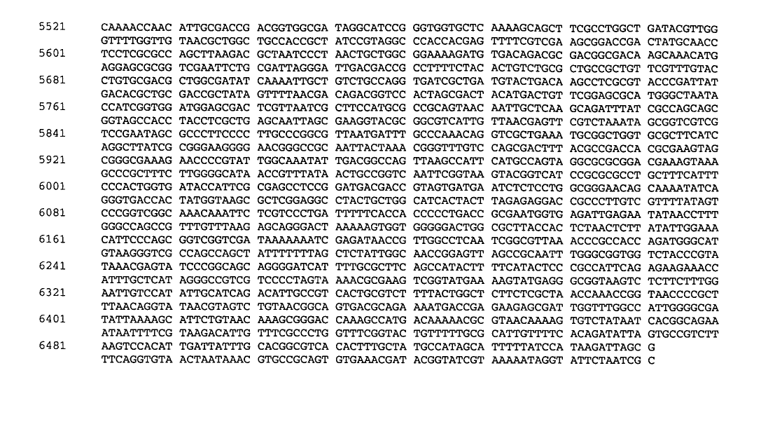

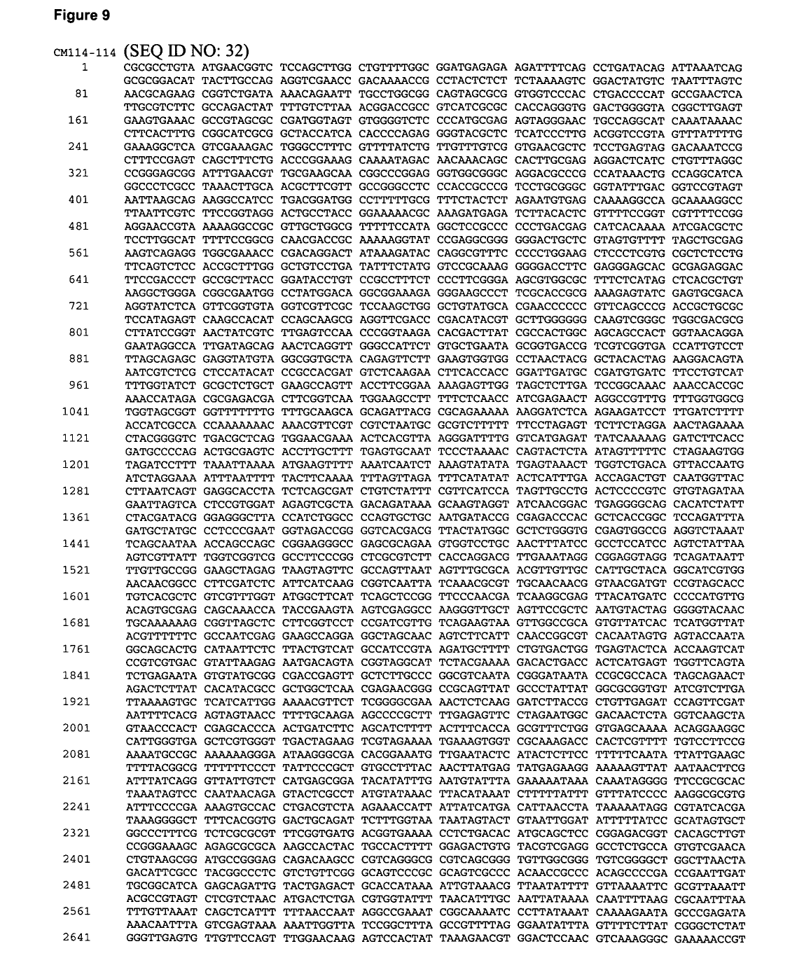

For the construction of a nucleic acid phage library, these fragments were digested with PstI and KpnI and ligated with T4 DNA ligase into the PstI and KpnI digested and alkaline phosphatase-treated phage display vectors CM114-iMab113 or CM114-iMab114 (FIGS. 7, 8 and 9). Cysteine-containing CDR3s were cloned into CM114-iMab114 while CDR3s without cysteines were cloned into vector CM114-iMab 113. The libraries were constructed by electroporation into E. coli TG1 electrocompetent cells by using a BTX electrocell manipulator ECM 630. Cells were recovered in SOB and grown on plates that contained 4% glucose, 100 micrograms ampicillin per milliliter in 2*TY-agar. After overnight culture at 37° C., cells were harvested in 2*TYmedium and stored in 50% glycerol as concentrated dispersions at −80° C. Typically, 5×10⁸ transformants were obtained with 1 μg DNA and a library contained about 10⁹ independent clones.

About 50 microliters of the library stocks was inoculated in 50 ml 2*TY/100 micrograms ampicillin/4% glucose and grown until an OD600 of 0.5 was reached. Next 10¹¹ VCSM13 (Stratagene) helper phages were added. The culture was left at 37° C. without shaking for 45 minutes to enable infection. Cells were pelleted by centrifugation and the supernatant was discarded. Pellets were resuspended in 400 ml 2*TY/100 micrograms ampicillin and cultured for one hour at 37° C. after which 50 μg/ml kanamycin was added. Infected cultures were grown at 30° C. for eight hours on a 200 rpm shaking platform. Next, bacteria were removed by pelleting at 5000 g at 4° C. for 30 minutes. The supernatant was filtered through a 0.45 micrometer PVDF filter membrane. Polyethyleneglycol and NaCl were added to the flow-through with final concentrations of, respectively, 4% and 0.5 M. In this way, phages were precipitated on ice and were pelleted by centrifugation at 6000 g. The phage pellet was solved in 50% glycerol/50% PBS and stored at −20° C.

The selection of phage-displayed VAPs was performed as follows. Approximately 1 μg of lactoferrin was immobilized in an immunotube (Nunc) or microtiter plate (Nunc) in 0.1 m sodium carbonate buffer (pH 9.4) at 4° C. o/n. After the removal of this solution, the tubes were blocked with a 1% BSA in PBS or a similar blocking agent for at least two hours either at room temperature or at 4° C. o/n. After removal of the blocking agent, a phagemid library solution containing approximately 10¹² to 10¹³ colony-forming units (cfu), which was preblocked with blocking buffer for one hour at room temperature, was added in blocking buffer. Incubation was performed on a slow rotating platform for one hour at room temperature. The tubes were then washed three times with PBS, two times with PBS with 0.1% Tween and again four times with PBS. Bound phages were eluted with an appropriate elution buffer, either 300 μl 0.1 m glycine pH 2.2 or 500 μl 0.1% trypsin in PBS. Recovered phages were immediately neutralized with 700 μl 1 M Tris-HCl pH 8.5 if eluted with glycine. Alternatively, the bound phages were eluted by incubation with PBS containing the antigen (1 to 10 μM). Recovered phages were amplified as described above employing E. coli XLI-Blue (Stratagene) or Top10F (InVitrogen) cells as the host. The selection process was repeated, mostly two to three times to concentrate positive clones. After the final round, individual clones were picked and their binding affinities and DNA sequences were determined. After subcloning as a NdeI-SfiI fragment into expression vector CM126, E. coli BL21 (DE3) or Origami (DE3) (Novagen) were transformed by electroporation and transformants were grown in 2×TY medium supplemented with Ampicillin (100 μg/ml). When the cell cultures reached an OD600, ˜1 protein expression was induced by adding IPTG (0.2 mM) or 2% lactose. After four hours at 37° C., cells were harvested by centrifugation.

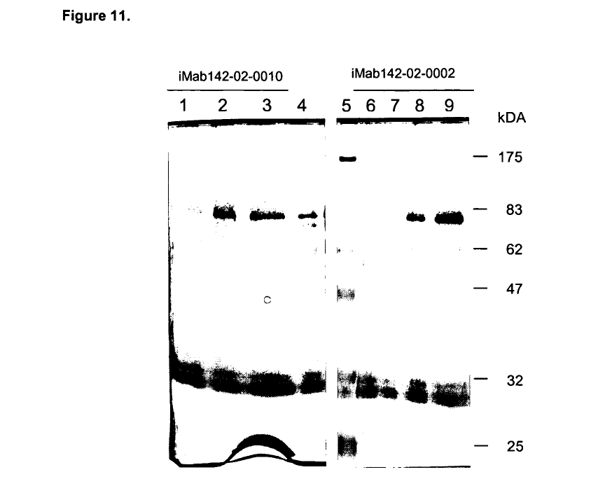

Proteins were isolated as described in Examples 4, 5 and 6 and binding to lactoferrin was performed as described in Example 10. The three nine-beta-stranded iMabs that bind lactoferrin specifically (FIGS. 10 and 11) are iMab142-02-0002, iMab142-02-0010 and iMab142-02-0011.

### Example 10

### Purification of Lactoferrin from Casein Whey by Affinity Chromatography

Lactoferrin from casein whey can be purified using direct affinity chromatography. IMab molecules with specific affinity against lactoferrin can be immobilized to nickel-nitrilo acetic acid (Ni-NTA)-agarose resin and subsequently exposed to casein whey. After washing, specifically bound lactoferrin can be eluted using a NaCl gradient.

Immobilization of iMab142-02-0002 and iMab100

Purified iMab142-02-0002 (with specific affinity against lactoferrin) and iMab100 (with specific affinity against lysozyme) were immobilized by metal affinity chromatography via specific binding of the 6*His affinity tag to nickel-nitrilo acetic acid (Ni-NTA)-agarose resin. Either iMab142-02-0002 or iMab100-02-0001 (25 mg) was mixed with 25 ml Ni-NTA-superflow resin, incubated for one hour in 10 mM phosphate buffer+137 mM NaCl (PBS) pH 8, packed in a column, washed with PBS pH 8+20 mM imidazole and subsequently equilibrated with PBS pH 6.5+20 mM imidazole. Imidazole is added to eliminate a specific binding of proteins to the Ni-NTA resin.

Preparation of Casein Whey from Fresh Milk

Fresh cow milk was heated up to 35° C. and acidified with H₂SO₄ (30%) to pH 4.6. The precipitated milk solution was centrifuged (12,000 rpm, 30 minutes) to remove solids. The supernatant was adjusted to pH 6.5 and further clarified by ultracentrifugation (25,000 rpm, 30 minutes) and filtration (0.45 μm filter).

Purification of Lactoferrin

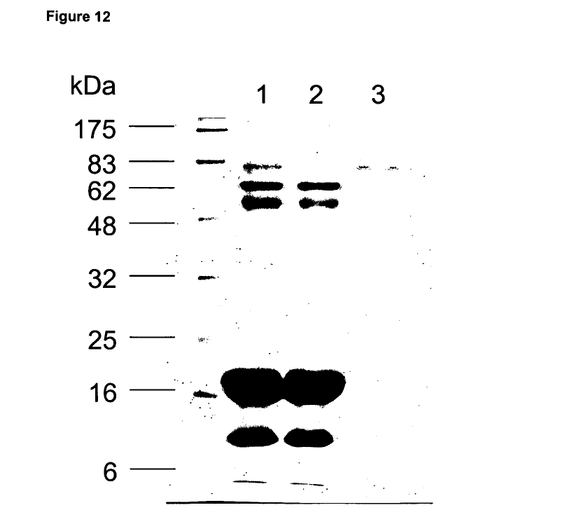

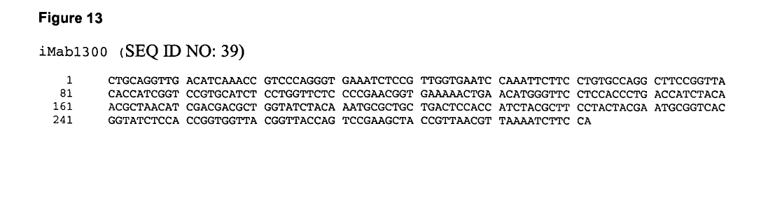

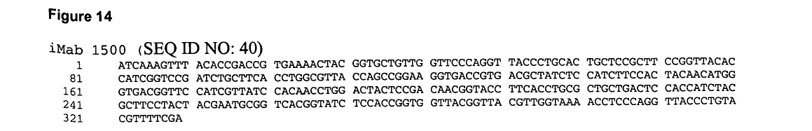

Clarified casein whey (50 ml, in PBS pH 6.5+20 mM imidazole) is loaded on an equilibrated Ni-NTA column immobilized with iMab142-02-0002. After loading, the column is washed with ten-column volumes of PBS pH 6.5+20 mM imidazole to remove non-specifically bound proteins. After washing, a linear NaCl gradient (0 to 1 M NaCl in PBS pH 6.5+20 mM imidazole) is applied to elute specific bound proteins. All steps were performed at a flow rate of 10 ml/minute. Fractions (flow-through and eluate) are collected and analyzed using SDS-PAGE (FIG. 12).

A single protein band of ˜80 kD was found in the eluate and was found to correspond to lactoferrin, which was evidenced by HPLC analysis using pure lactoferrin as reference.

As negative controls, a Ni-NTA matrix (25 ml) without immobilized iMab, and a Ni-NTA matrix with immobilized iMab100 (with specific affinity to lysozyme) was used. No significant binding of lactoferrin was found in any of the two controls indicating that the purified lactoferrin does not result from a specific binding to the resin nor to a specific binding to the iMab scaffold.

### Example 11

### Purification of Lactoferrin from Casein Whey by Indirect Affinity Chromatography

Lactoferrin from casein whey can be purified using indirect affinity chromatography. IMab molecules with specific affinity against lactoferrin are mixed with casein whey (in PBS pH 6.5+20 mM imidazole) and incubated for an hour under continuous stirring to allow binding. Subsequently, the iMab molecules (whether bound to lactoferrin or not) can be immobilized by metal affinity chromatography via specific binding of the 6*His affinity tag to nickel-nitrilo acetic acid (Ni-NTA)-agarose resin. The immobilized resin can be packed in a column, washed with PBS pH 6.5+20 mM imidazole to remove non-specifically bound proteins. The bound lactoferrin can be eluted using a NaCl gradient.

### Example 12

### Isolation and Identification of Lactoferrin Binding Seven-Beta-Stranded iMabs

In order to be able to subclone amplified affinity regions into these iMabs for the construction of a nucleic acid phage display library having variegations in AR4, restriction sites were designed around the AR4 region of iMab1300 and 1500. For iMab1500, HindIII and EcoRI sites were introduced, while for iMab1300, PstI and HindIII sites were introduced, resulting in iMab143-02-0003 and iMab144-02-0003, respectively. iMab143-02-0003 and iMab144-02-0003 were constructed. The resulting iMabs were cloned in frame into CM114 (FIG. 7) as a NotI-SfiI fragment. Llama CDR3 regions were amplified as described above, except that in order to adapt the environment of the camelidae CDR3 regions to these scaffold primers, three extra rounds of PCR similar to the first PCR method was performed on 5 μl of the products with the exception that the cycle number was decreased to 15 cycles. For iMab143-02-0003, primers 813 and 814 (Table 4) were subsequently used as forward primer and primers 815, 816 and 817 (Table 4) were used as reverse primer. For iMab144-02-0003, primers 822, 823 and 824 were subsequently used as forward primer and primers 829, 811 and 830 were used as reverse primer. After digestion with the appropriate restriction enzymes, the fragments were cloned into the phage display vector CM114 (see FIG. 7).

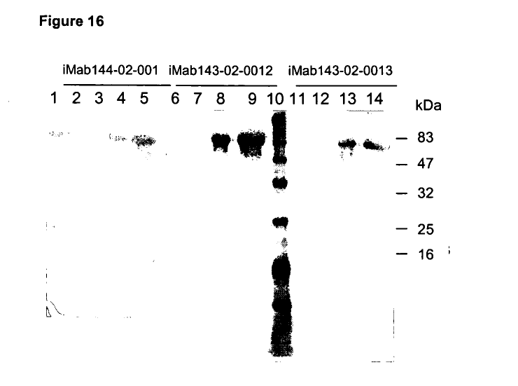

Selection for lactoferrin binding iMabs was performed as described in Example 9. Three seven-beta-stranded iMabs that bind lactoferrin specifically were isolated, being iMab143-02-0012, iMab143-02-0013 and iMab144-02-0014 (FIG. 10). Proteins were produced and purified and binding was tested as described in Example 10. The results are shown in FIG. 16.

### Example 13

### Covalent Immobilization of iMab Molecules to Pre-Activated Supports

Purified iMab can be covalently coupled to pre-activated matrices, such as Eupergit or Sepabeads, which exhibit excellent physical and chemical stability to perform under harsh industrial conditions.

Purified iMab can be directly and covalently bound to supports with epoxy groups while the affinity for the target molecule is retained.

Eupergit (Röhm) or Sepabeads (Mitsubishi) (1 g) is mixed with 10 to 50 mg iMab in 10 ml binding buffer (0.5 to 1.0 M KPO₄ buffer pH 8 to 10). After overnight stirring at room temperature, resin is washed excessively with binding buffer and afterwards blocked with 10 ml 0.2 M ethanolamine in binding buffer. Alternatively, mercaptoethanol, glycine or Tris can be used as blocking agent. After four hours stirring at room temperature, the immobilized resin is washed twice with binding buffer.

Purified iMab can also be covalently coupled to supports with primary amino groups while the affinity for the target molecule is retained. The reaction involves generation of aldehyde groups using glutaraldehyde and sodium cyanoborohydride prior to iMab immobilization.

Sepabeads (100 ml) containing amine groups are washed with coupling buffer (0.05 M to 0.5 M NaPO₄ buffer, 0.05 to 0.5 M NaCl pH 6 to 8) and incubated in 100 ml 5 to 25% glutaraldehyde (w/v)+0.6 g NaCNBH₃ in coupling buffer for at least 4 hours (room temperature). After excessive washing of the activated matrix with coupling buffer, the beads are incubated in 100 ml of iMab (1 to 20 mg/ml) dissolved in coupling buffer. After addition of 0.6 g NaCNBH₃, the mixture is stirred for at least four hours at room temperature, washed with coupling buffer, water, NaCl (1 M) and water.

### Example 14

### Correct Orientation of iMab Molecules to Pre-Activated Supports

Pre-activated supports with aldehyde or epoxy groups predominantly react with the amine side chain of lysine residues. To promote correct orientation of the immobilized iMab, a lysine-rich tail comprising two to four lysines is modeled at the C-terminus of the iMab molecule, which is far exposed from the affinity regions. IMab scaffolds with three different lysine tails have been constructed (as shown below) of which all can be covalently bound to pre-activated resin.

Lysine tail (short) ASSAGSKGSK (SEQ ID NO: 1)

Lysine tail (medium) ASSAFGSKGKSK (SEQ ID NO: 2)

Lysine tail (long) ASSAGSKGKSKGSK (SEQ ID NO: 3)

Moreover, as a next step to prevent incorrect positioning of the iMab to the resin, the iMab scaffold is modeled such that all residual lysines in the scaffold have been replaced by other amino acids without changing the structure, solubility or stability of the protein. A modeled amino acid sequence of iMab100 without any lysines in the scaffold is shown below.

IMab molecules with a lysine tail (short, medium or long) but without any other lysines in the scaffold can be positioned correctly to a preactivated resin after which the affinity to the target molecule is retained.

Table 1: Protein sequences for VAPs with affinity for chicken lysozyme and different pI. iMab100 was used as a template for the design of scaffolds that differ in pHi and external amino acids. iMab135-02-0001, iMab136-02-0001 and iMab137-02-0001 are example results of functional scaffolds that bind and fold correctly but differ in pl.

Table 2: Determination of pI values of iMabs. Prosa-II scores, measured and calculated pI values of individual iMabs.

Table 3: Titration curves of four different iMabs. Theoretical determination of the titration curves of iMab100, iMab135-02-0001, iMab137-02-0001 and iMab136-02-0001 (including tags).

Table 4: Primer sequences.

TABLE 1

| | | |
|---|---|---|
| 1 | 50 | |
| iMab100 + VSV + HIS | MNVKLVEK-GGNFVENDDDLKLTCRAEGYTIGPYCMGWFRQAPNDDSTNV | (SEQ ID NO: 4) |
| iMAB135-02-0001 | MNVQLVES-GGNFVENDQDLSLTCRASGYTIGPYCMGWFRQAPNQDSTGV | (SEQ ID NO: 5) |
| iMAB136-02-0001 | MNVKLVEK-GGNFVENDDDLRLTCRAEGYTIGPYCMGWFRQAPNRDSTNV | (SEQ ID NO: 6) |
| iMAB137-02-0001 | MNVQLVES-GGNFVENDQSLSLTCRASGYTIGPYCMGWFRQAPNSRSTGV | (SEQ ID NO: 7) |
| Consensus | MNV.LVE. GGNFVEND..L.LTCRA.GYTIGPYCMGWFRQAPN.DST.V | (SEQ ID NO: 8) |
| 51 | 100 | |
| iMab100 + VSV + HIS | ATINMGGGITYYGDSVKERFDIRRDNASNTVTLSMDDLQPEDSAEYNCAG | |
| iMAB135-02-0001 | ATINMGGGITYYGDSVKERFRIRRDNASNTVTLSMQNLQPQDSANYNCAA | |
| iMAB136-02-0001 | ATINNGGGITYYGDSVKERFDIRRDNASNTVTLSMTNLQPSDSASYNCAA | |
| iMAB137-02-0001 | ATINMGGGITYYGDSVKGRFTIRRDNASNTVTLSMNDLQPRDSAQYNCAA | |
| Consensus | ATINMGGGITYYGDSVKERF.IRRDNASNTVTLSM..LQP.DSA.YNCA | |
| 101 | 150 | |
| iMab100 + VSV + HIS | DSTIYASYYECGHGLSTGGYGYDSHYRGQGTDVTVSSASSAGGGGSYTDI | |
| iMAB135-02-0001 | DSTIYASYYECGHGLSTGGYGYDS--RGQGTSVTVSSASSAGGGGSYTDI | |
| iMAB136-02-0001 | DSTIYASYYECGHGLSTGGYGYDS--RGQGTRVTVSSASSAGGGGSYTDI | |
| iMAB137-02-0001 | DSTIYASYYECGHGLSTGGYGYDS--RGQGTDVTVSSASSAGGGGSYTDI | |
| Consensus | DSTIYASYYECGHGLSTGGYGYDS RGQGT.VTVSSASSAGGGGSYTDT | |
| 151 | 165 | |
| iMab100 + VSV + HIS | EMNRLGKSHHHHHHG | |
| iMAB135-02-0001 | EMNRLGKSHHHHHHG | |
| iMAB136-02-0001 | EMNRLGKSHHHHHHG | |
| iMAB137-02-0001 | EMNRLGKSHHHHHHG | |
| Consensus | EMNRLGKSHHHHHHG | |

TABLE 2
Determination of Isoelectric point (pI)

| | Prosa-II scores | pI measured | pI Calculated |
|---|---|---|---|
| iMab137-02-0001 | −6.78 (no tag) | 7.5 | 6.68 |
| iMab136-02-0001 | −6.58 (no tag) | 7.0 | 6.43 |
| iMab135-02-0001 | −6.59 (no tag) | 7.0 | 6.20 |
| iMab100 with VSV + HIS | | 6.2 | 4.86 |

[image] [image] TABLE 4

| | |
|---|---|
| Primer | Sequence |

| | |
|---|---|
| number | 5′ → 3′ |

| | |
|---|---|
| Pr8 | CCTGAAACCTGAGGACACGGCC |
| | (SEQ ID NO: 9) |
| Pr9 | CAGGGTCCCC/TTG/TGCCCCAG |
| | (SEQ ID NO: 10) |
| Pr16 | CCACRTCCACCACCACRCAYGTGACCT |
| | (SEQ ID NO: 11) |
| Pr49 | GGTGACCTGGGTACCC/TTG/TGCCCCGG |
| | (SEQ ID NO: 12) |
| Pr56 | GGAGCGC/TGAGGGGGTCTCATG |
| | (SEQ ID NO: 13) |
| Pr73 | GAGGACACTGCCGTATATTAC/TTG |
| | (SEQ ID NO: 14) |
| Pr75 | GAGGACACTGCAGAATATAAC/TTG |
| | (SEQ ID NO: 15) |
| Pr76 | CCAGGGAAGG/CAGCGC/TGAGTT |
| | (SEQ ID NO: 16) |
| Pr811 | GACCTGGGTCCCAGG/TTTCCCA |
| | (SEQ ID NO: 17) |
| Pr813 | GAGGACACGGCAGGT/CTATAAC/TTG |
| | (SEQ ID NO: 18) |
| Pr814 | GAGGACACGGAAAGCTTTACC/TTG |
| | (SEQ ID NO: 19) |
| Pr815 | CGGTGACCTGGGTCCCC/TGG/TGTCCCAG |
| | (SEQ ID NO: 20) |
| Pr816 | CGGTGACCTGGGTCCCC/TGG/TATCCCCG |
| | (SEQ ID NO: 21) |
| Pr817 | CGGTGACCTGGGTCCCC/TGA TTCCCG |
| | (SEQ ID NO: 22) |
| Pr822 | CCTGAGGACGCGGCCATT/CTATTAC/TTG |
| | (SEQ ID NO: 23) |
| Pr823 | CCTGAGGCCGCAGGCATT/CTATTAC/TTG |
| | (SEQ ID NO: 24) |
| Pr824 | CCTGAGGCTGCAGGCATT/CTATAAC/TTG |
| | (SEQ ID NO: 25) |
| Pr829 | CGGTGACCTGGGTCCCC/TG/TTCCCCA |
| | (SEQ ID NO: 26) |
| Pr830 | CGGTGACCTGGGTCCAAGCTTCCGA |
| | (SEQ ID NO: 27) |

1. A method for at least in part isolating a particular compound from its environment, said method comprising:
selecting a proteinaceous binding molecule with a binding specificity for said compound,
modifying said proteinaceous molecule such that the pKi of said proteinaceous molecule in an aqueous medium is altered when compared to the pKi of the original proteinaceous binding molecule, wherein said modification results in a reduction of binding of an undesired compound from said environment to the thus altered proteinaceous binding molecule,
providing said altered proteinaceous molecule to said environment to allow binding of said particular compound, and
separating said altered proteinaceous molecule from said environment.

2. The method according to claim 1, wherein said proteinaceous binding molecule is altered through amino acid substitution.

3. The method according to claim 1, wherein said proteinaceous molecule comprises an immunoglobulin or a functional part, derivative and/or analogue thereof.

4. The method according to claim 1, wherein said proteinaceous molecule comprises a synthetic or recombinant proteinaceous molecule comprising a binding peptide and a core, said core comprising a b-barrel comprising at least 4 strands, wherein said b-barrel comprises at least two β-sheets, wherein each of said β-sheets comprises two of said strands and wherein said binding peptide is a peptide connecting two strands in said b-barrel and wherein said binding peptide is outside its natural context.

5. The method according to claim 1, further comprising: separating said altered proteinaceous binding molecule bound compound and collecting said compound.

6. The method according to claim 1, wherein said environment comprises a biological product.

7. The method according to claim 1, wherein said modification is at a surface that is exposed to said proteinaceous binding molecule's exterior.

8. The method according to claim 1, wherein said modification is not in the proteinaceous binding molecule's compound binding part.

9. A proteinaceous binding molecule comprising:
a binding peptide, and
a core for at least partly isolating a particular compound from its environment,
wherein said proteinaceous binding molecule is adapted for improved binding specificity of said compound in said environment.

10. The proteinaceous binding molecule of claim 9, wherein said adaptation comprises a modification of the core of said proteinaceous binding molecule.

11. The proteinaceous binding molecule of claim 10, wherein said core is modified on a surface that is exposed to the proteinaceous binding molecule's exterior.

12. The proteinaceous binding molecule of claim 9, wherein said adaptation comprises an altered pKi.

13. The proteinaceous binding molecule of claim 9, wherein said adaptation results from an amino acid substitution.

14. The proteinaceous binding molecule of claim 9, wherein said proteinaceous binding molecule comprises:
a synthetic or recombinant proteinaceous molecule comprising a binding peptide and a core, said core comprising a b-barrel comprising at least 4 strands, wherein said b-barrel comprises at least two β-sheets, wherein each of said β-sheet comprises two of said strands and wherein said binding peptide is a peptide connecting two strands in said b-barrel and wherein said binding peptide is outside its natural context.

15. The proteinaceous binding molecule of claim 9, comprising a sequence or encoded by a sequence as depicted in FIG. 10, FIG. 15 or Table 1 or a functional part, derivative and/or analogue thereof.

16. The proteinaceous binding molecule of claim 15, wherein at least part of the binding peptide is removed.

17. The proteinaceous binding molecule of claim 15 further provided with a different specific binding peptide.

18. The proteinaceous binding molecule of claim 9, wherein said adaptation comprises adding or removing an amino acid exposed to the proteinaceous binding molecule's exterior, wherein said amino acid is able to chemically link with a carrier surface.

19. The proteinaceous binding molecule of claim 18, wherein said amino acid comprises a reactive amino or carboxyl group.

20. The proteinaceous binding molecule of claim 19, wherein said amino acid comprises glycine.

21. The proteinaceous binding molecule of claim 9, having a binding specificity for a lactoferrin form, a lactoperoxidase, a growth factor, an antibody, a lysozyme, or an oligosaccharide, a lipid, biotin, a viral protein, a bacterial toxin, and/or a bacterial surface marker.
